(19) United States
(12) Patent Application Publication
(10) Pub. No.: US 2024/0016875 A1
(43) Pub. Date: Jan. 18, 2024

(54) SHAN-ZHA FOR THE TREATMENT OF DEPRESSION AND ANXIETY DISORDERS

(71) Applicant: THE OPEN UNIVERSITY, Raanana (IL)

(72) Inventor: Ravid DORON, Tel Aviv (IL)

(21) Appl. No.: 18/008,996

(22) PCT Filed: Jun. 8, 2021
(86) PCT No.: PCT/IL2021/050685

§ 371 Date: Dec. 8, 2022

(30) Foreign Application Priority Data
Jun. 8, 2020 (IL) 275222

(51) Int. Cl. A61K 36/734; A61P 25/22; A61P 25/24

(52) U.S. Cl. CPC A61K 36/734; A61P 25/22; A61P 25/24; A61K 2236/333; A61K 2236/51

(57) ABSTRACT

The invention concerns pharmaceutical and nutritional compositions comprising hawthorn fruit (shan za), or an active fraction extracted therefrom, for the treatment or alleviation of symptoms of anxiety disorders, stress, or depression.

### TECHNOLOGICAL FIELD

The present invention relates to the use of the Chinese herb Shan-zha and extracts thereof for treating depression and anxiety disorders.

### BACKGROUND ART

References considered to be relevant as background to the presently disclosed subject matter are listed below:
[1] Katzman, M. A., Current considerations in the treatment of generalized anxiety disorder. CNS Drugs, 2009. 23(2): p. 103-20.
[2] Reinhold, J. A., et al., Pharmacological treatment of generalized anxiety disorder. Expert Opin Pharmacother, 2011. 12(16): p. 2457-67.
[3] Farach, F. J., et al., Pharmacological treatment of anxiety disorders: current treatments and future directions. J Anxiety Disord, 2012. 26(8): p. 833-43.
[4] Linde, K., M. M. Berner, and L. Kriston, St John's wort for major depression. Cochrane Database Syst Rev, 2008(4): p. CD000448.
[5] Doron, R., et al., Anxiolytic effects of a novel herbal treatment in mice models of anxiety. Life Sci, 2012. 90(25-26): p. 995-1000.
[6] Doron, R., et al., Escitalopram or novel herbal mixture treatments during or following exposure to stress reduce anxiety-like behavior through corticosterone and BDNF modifications. PLoS One, 2014. 9(4): p. e91455.
[7] Doron, R., et al., A novel herbal treatment reduces depressive-like behaviors and increases BDNF levels in the brain of stressed mice. Life Sci, 2014. 94(2): p. 151-7.
[8] Autry, A. E. and L. M. Monteggia, Brain-derived neurotrophic factor and neuropsychiatric disorders. Pharmacol Rev, 2012. 64(2): p. 238-58.
[9] Butterweck, V., M. Hegger, and H. Winterhoff, Flavonoids of St. John's Wort reduce HPA axis function in the rat. Planta Med, 2004. 70(10): p. 1008-11.
[10] Prenner, L., et al., Reduction of high-affinity 02-adrenergic receptor binding by hyperforin and hyperoside on rat C6 glioblastoma cells measured by fluorescence correlation spectroscopy. Biochemistry, 2007. 46(17): p. 5106-5113.
[11] Doron R. et al., GABA_{A} Receptor Density Is Not Altered by a Novel Herbal Anxiolytic Treatment, Journal of Molecular Neuroscience 65, 110-117 (2018).
[12] Doron R. et al., Cerebral MAO Activity Is Not Altered by a Novel Herbal Antidepressant Treatment, Journal of Molecular Neuroscience 69, 371-379 (2019).
[13] Avitsur R. et al Escitalopram or novel herbal treatments differentially alter cytokine and behavioral responses to immune challenge, Journal of Neuroimmunology, 309, 111-118 (2017).
[14] Jurikova, T., et al., Polyphenolic profile and biological activity of Chinese hawthorn (Crataegus pinnatifida BUNGE) fruits. Molecules, 2012. 17(12): p. 14490-14509.
[15] Morozova T. V. et al., Antidepressant activity of redhaw hawthorn (crataegus sanguinea) extracts. PHARMACY, 2017. Volume 66, 4, p/3-39.
[16] Kurkin, V. A. et al., Comparative Investigation of the Diurethic and Antidepressant Activity of Liquid Extracts of Crataegus Sanguinea. Journal of Pharmaceutical Sciences and Research, 2019. Vol. 11, Iss. 1, p. 30-32.
[17] Popovic-milenkovic M. T. et al., Antioxidant and anxiolytic activities of crataegus nigra wald. et kit. berries. Acta Poloniae Pharmaceutica—Drug Research, 2014. Vol. 71 No. 2 p. 279-285.
[18] Devrim Can O. et al., Effects of hawthorn seed and pulp extracts on the central nervous system. Pharmaceutical Biology, 2010. Vol. 48:8, p. 924-931.
[19] Chun L. et al., A novel herbal treatment reduces depressive-like behaviors and increases brain-derived neurotrophic factor levels in the brain of type 2 diabetic rats. Neuropsychiatr Dis Treat. 2016; Vol. 12: p. 3051-3059.

Acknowledgement of the above references herein is not to be inferred as meaning that these are in any way relevant to the patentability of the presently disclosed subject matter.

### BACKGROUND

Depression and anxiety disorders are highly prevalent and considered of major public health concern worldwide. According to the World Health Organization (WHO), depression is projected to be the second most prevalent cause of illness-induced disability by 2020 and the largest contributor to disease burden by 2030. Depression and anxiety disorders are characterized by frequent comorbidity with each other as well as with other mental and physical disorders. In addition, the societal cost of the physical, mental, and broader personal difficulties associated with these disorders is substantial. Despite the availability of a wide range of drugs for treating depression and anxiety, most patients fail to achieve complete and sustained remission of symptoms. Selective serotonin reuptake inhibitors (SSRIs) and serotonin-norepinephrine reuptake inhibitors (SNRIs), the current first-line treatment for depression and anxiety, are characterized by low success rate and wide variety of side effects, including sexual dysfunction and weight gain [1-3].

The limitations of existing conventional drugs have prompted the search for alternative pharmacotherapies for anxiety and depression such as herbal medicines. One available herbal medicine is the St. John's Wort, also known as Hypericum [4]. Compounds isolated from St. John's Wort were shown to down regulate the hypothalamus-pituitary-adrenal (HPA) axis function [9], to reduce stress-induced noradrenergic response in glial cells [10].

U.S. Pat. No. 9,320,772 discloses a novel herbal treatment (NHT) which efficaciously reduced anxiety and depressive-like behaviors in stressed mice in a similar manner to the SSRI escitalopram [5-7]. NHT-induced anxiolytic- and antidepressant-like effects were associated with elevations in brain-derived neurotrophic factor (BDNF) levels in the hippocampus and prefrontal cortex (PFC) as well as reductions in circulating corticosterone levels [5-7]. BDNF is known to play a key role in the pathophysiology of depression and anxiety and is essential for the response to antidepressants [8]. As opposed to escitalopram, NHT did not cause sexual dysfunction and did not reduce serotonin transporter (SERT) levels in the PFC. In addition, NHT does not alter GABA_{A} receptor density [11] or cerebral MAO (monoamine oxidase) activity [12], and it alters cytokine and behavioral responses to immune challenge [13].

NHT consists of identical amounts of four herbs: Shan-zha (i.e., Crataegus pinnatifida), Fu-xiao-mai (i.e., Triticum aestivum), Baihe (i.e., Lilium brownie) and Da-zao (i.e., Fructus Zizyphi Jujubae). Chinese hawthorn (Crataegus pinnatifida Bge.) fruits were shown to be effective in lowering blood cholesterol and the risk of cardiovascular diseases and were also shown to hold antioxidant and anti-inflammatory capacities [14].

Redhaw hawthorn (crataegus sanguinea) extracts showed antidepressant activity [15, 16]. Crataegus nigra wald. et kit. Berries showed antioxidant and anxiolytic activities [17]. Crataegus monogyna Jacq. (Rosaceae) pulp and seed extracts were shown to have CNS depressant activities on mice, reducing exploratory behavior, locomotor activities as well as analgesic activities [18]. A combination treatment of Radix Puerariae and hawthorn fruit was shown to reduce depressive-like behavior in diabetic rats [19].

None of these publications describe anxiolytic and/or antidepressant activities of Shan-zha (Crataegus pinnatifida) in humans.

### GENERAL DESCRIPTION

In a first of its aspects, the present invention provides a pharmaceutical or nutritional composition, comprising hawthorn fruit (shan za), or an active fraction extracted thereof, as the active agent, for the treatment or alleviation of symptoms of anxiety disorders, stress, or depression in a subject.

In one embodiment, said fraction is an ethanol fraction.

In one embodiment, said ethanol fraction is a 10%, 20%, 50% or 70% ethanol fraction.

In one embodiment, said ethanol fraction is a 50% ethanol fraction.

In one embodiment, said ethanol fraction is a 70% ethanol fraction.

In one embodiment, said ethanol fraction is prepared using high-performance liquid

chromatography (HPLC).

In one embodiment, said ethanol fraction is prepared by a method comprising:
a. homogenizing Shan-zha plant powder in 20% ethanol;
b. stirring the Shan-zha —ethanol mixture thereby obtaining an ethanol extract;
c. centrifuging the ethanol extract;
d. applying the supernatant to a separation column;
e. eluting the column with increasing concentrations of ethanol; and
f. freeze-drying the eluted fractions;
thereby obtaining ethanol fractions of Shan-zha.

In one embodiment, said step b is performed at a temperature range of 30-80° C.

In one embodiment, said step d is performed by filling the entire separation column.

In one embodiment, the composition further comprises one or more of an oil solvent, DMSO, an antioxidant, a vitamin, an inert carrier, a stabilizer or a surfactant.

In one embodiment, said composition does not comprise any additional herbal components other than hawthorn fruit or an active fraction extracted thereof.

In one embodiment, the composition is formulated to be suitable for oral, local, or parenteral administration.

In one embodiment, said composition is in the form selected from the group consisting of a tablet, a capsule, a liquid, syrup, tincture, powder, granules (e.g., freeze-dried granules) and raw herbs decoction.

In one embodiment, said composition is encapsulated within a microcapsule.

In one embodiment, said microcapsule is a liposome or a micelle.

In one embodiment, said composition does not cause weight gain by said treated subject and/or does not result in reduction of sexual function of said treated subject.

In another aspect, the present invention provides a method of treating or alleviating symptoms of anxiety disorders, stress or depression comprising administering to a patient in need thereof an effective amount of a composition according to the invention, wherein said amounts are effective to treat or alleviate symptoms of anxiety disorders, stress or depression.

In one embodiment, the amount of the composition administered is between about 1 g/day to about 15 g/day, between about 2 g/day to about 3 g/day, or about 2.5 g/day, or about 10 g/day.

In one embodiment, the amount of the composition administered is between about 1 mg/kg to 100 mg/kg, between about 2 mg/kg to 50 mg/kg or between about 3 mg/kg to 30 mg/kg.

In one embodiment, administration of said composition causes an increase in the level of BDNF in the hippocampus and prefrontal cortex (PFC) of the treated patient, and/or does not reduce serotonin transporter (SERT) levels in the PFC of the treated patient.

In one embodiment, the administration of said composition does not affect the weight or the sexual function of the treated patient.

In one embodiment, composition is suitable for treating breast feeding women.

In one embodiment, said treated patient is a breast-feeding woman.

In one embodiment, the efficiency of the treatment is measured by a test selected from the group consisting of the Hamilton depression rating scale (HAM), Clinical Global Impression (CGI), Sheehan Disability Scale (SDS), and a combination thereof.

In one embodiment, the composition is administered for 3 weeks.

In another aspect, the present invention provides a pharmaceutical or nutritional composition, comprising hawthorn fruit (shan za), or an active fraction extracted thereof, as the active agent, for use in a method of treating or alleviating symptoms of anxiety disorders, stress, or depression.

In another aspect, the present invention provides a method for preparing an ethanol fraction of hawthorn fruit (shan za), comprising:
a. homogenizing Shan-zha plant powder in 20% ethanol;
b. stirring the Shan-zha —ethanol mixture thereby obtaining an ethanol extract;
c. centrifuging the ethanol extract;
d. applying the supernatant to a separation column;
e. eluting the column with increasing concentrations of ethanol; and
f. freeze-drying the eluted fractions;
thereby obtaining ethanol fractions of Shan-zha.

In one embodiment, step b is performed at a temperature range of 30-80° C.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1a-1c. The behavioral effect of treatment with NHT's herbal constituents.

FIG. 1a: Treatment with Shan-zha (N=16) and Baihe (N=15) reduced anxiety-like behavior in the EPM compared to vehicle (N=15), similarly to escitalopram (N=15) and NHT (N=17).

FIG. 1b: Treatment with Shan-zha (N=16) reduced depressive-like behavior in the Tail suspension test (TST) compared to vehicle (N=12), similarly to escitalopram (N=14) and NHT (N=16).

FIG. 1c: No change in locmotor activity was induced by NHT herbal constituents, similarly to escitalopram and NHT.

Results are presented as mean±SEM. *p<0.05 and **p<0.005 vs. vehicle.

FIG. 2a-2b. The effect of treatment with NHT's herbal constituents on brain BDNF levels in stressed mice.

FIG. 2a: Treatment with Shan-zha (N=5) elevated hippocampal BDNF levels compared to vehicle (N=8), similarly to escitalopram (N=7) and NHT (N=7).

FIG. 2b: Treatment with Shan-zha (N=5) and Baiha (N=4) elevated BDNF levels in the PFC compared to vehicle (N=6), similarly to escitalopram (N=6) and NHT (N=6).

Results are presented as mean±SEM. *p<0.05 and **p<0.005 vs. vehicle.

FIG. 3a-3c. The effect of treatment with Shan-zha on SERT levels, weight change and sexual function.

FIG. 3a: Treatment with Shan-zha (N=10) did not alter SERT levels in the PFC, while treatment with escitalopram (N=8) significantly reduced SERT levels compared to vehicle (N=9).

FIG. 3b: Treatment with Shan-zha (N=20) did not alter weight change, while treatment with escitalopram (N=18) significantly increased weight compared to vehicle (N=18).

FIG. 3c: Treatment with Shan-zha (N=20) did not alter sexual function, while treatment with escitalopram (N=18) significantly reduced sexual function compared to vehicle (N=20).

Results are presented as mean±SEM. *p<0.05 and **p<0.005 vs. vehicle.

FIG. 4: Diagram depicting experiment outline.

After mating dams were submitted to restrain stress or naïve conditions until parturition. On PND1, locomotor activity and anxiety-like behavior of dams were tested via the Open Field Test (OFT) and the EPM, respectively. Subsequently, dams were assigned to treatment with escitalopram, shan-zha or vehicle for three weeks. On PND21 dams and pups anxiety-like behavior was tested via the EPM. Shortly after, mice were submitted to cervical dislocation and prepared for neurochemical assessments. GD=gestational day; P=parturition; PND=postnatal day; CD=cervical dislocation.

FIG. 5a-5c: The effect of restrain stress on litter size, locomotor activity and anxiety-like behavior, in dams, on day 1 post-parturition.

FIG. 5a: No difference between the naïve group and the stress group was observed in litter size.

FIG. 5b: In the OFT, there was no difference between the naïve group and the stress group in locomotor activity.

FIG. 5c: In the EPM, the stress group demonstrated a significant reduction in percentage of time in open arms (i.e., anxiety-like behavior) compared to the naïve group. n=15 dams per group. ***p<0.001.

FIG. 6a-6c. The effects of restrain stress during gestation and pharmacological treatments on anxiety-like behavior, hippocampal BDNF concentration and free serum SERT, in dams, on day 21 post-parturition.

FIG. 6a: In the EPM, the stress-vehicle group demonstrated increased anxiety-like behavior compared to the naïve-vehicle, stress-escitalopram and stress-shan-zha groups.

FIG. 6b: Stressed dams demonstrated reduced hippocampal BDNF concentration compared to naïve dams.

FIG. 6c: Dams treated with escitalopram demonstrated reduced serum SERT free for binding compared to the vehicle- or shan-zha-treated dams, indicating increased serum escitalopram levels. n=4-5 dams per group. *p<0.05**p<0.01***p<0.001.

FIG. 7a-7c. The effects of prenatal stress and pharmacological treatments via lactation on anxiety-like behavior, hippocampal BDNF concentration and free serum SERT, in 21 days old pups.

FIG. 7a: In the EPM, the stress-vehicle group demonstrated increased anxiety-like behavior compared to the naïve-vehicle, stress-escitalopram and stress-shan-zha groups. n=35-38 pups per group.

FIG. 7b: Pups in the prenatal stress group demonstrated reduced hippocampal BDNF concentration compared to naïve pups. n=14-16 pups per group.

FIG. 7c: Pups treated with escitalopram via lactation demonstrated reduced serum SERT free for binding compared to the vehicle or the shan-zha treatment groups, indicating increased serum escitalopram levels. n=14-16 pups per group. *p<0.05**p<0.01****p<0.0001.

FIG. 8a: Ckb gene upregulated after the UCMS and downregulated by treatments. From left to right: Escitalopram, None, Saline, Shan-zha.

FIG. 8b: rab11B gene upregulated after the UCMS and downregulated by treatments. From left to right: Escitalopram, None, Saline, Shan-zha.

FIG. 9a: IGSF9B gene (immunoglobulin superfamily member 9B) differently changed in the Shan-zha treatment group.

FIG. 9b: Mif gene (macrophage migratory inhibitory factor (glycosylation inhibiting factor)) differently changed in the Shan-zha treatment group.

FIG. 9c: NDUFA3 gene (NADH-ubiquinone oxidoreductase subunit A3) differently changed in the Shan-zha treatment group.

FIG. 9d: Hint2 gene (histidine triad nucleotide binding protein 2) differently changed in the Shan-zha treatment group.

FIG. 10: Effect of Shan-zha treatment on depression symptoms.

Change in HAM-D was calculated as the D between each visit. Two-ways ANOVA with repeated measurements: Treatment X Week; p<0.005* p<0.05**p<0.01.

FIG. 11: Schematic representation of Study Timeline of Clinical study.

FIG. 12: Schematic representation of Proportion of dropout categorized by treatment group and trial period.

FIG. 13: Graph showing difference in time to remission between treatment groups (Shan-Zha/placebo). Asterisk sign indicates significance level of <0.05. Error bars: +/−1 SD.

FIG. 14a-14b: Depression scores following Shan zha treatment versus placebo.

FIG. 14a: Graph showing mean of HAM-A scores between treatment groups (Shan zha/placebo) throughout the seven meeting. Asterisk sign indicates significance level of <0.05. Error bars: +/−1 SD.

FIG. 14b: Group showing mean of HAM-D scores between treatment groups (Shan zha/placebo) throughout the seven meeting. Asterisk sign indicates significance level of <0.05. Error bars: +/−1 SD.

FIG. 15a-15b: Social scores following Shan zha treatment versus placebo.

FIG. 15a: Graph showing mean of SDS-S scores between treatment groups (Shan zha/placebo) throughout the seven meeting. Asterisk sign indicates significance level of <0.05. Error bars: +/−1 SD.

FIG. 15b: Graph showing mean of SDS-S scores between treatment groups (Shan zha/placebo) between time 5 and time 6. Asterisk sign indicates significance level of <0.05. Error bars: +/−1 SD.

FIG. 16a-16c: Sexual scores following Shan zha treatment versus placebo.

FIG. 16a: Graph showing mean changes in score of question 12 of HAM-A between treatment groups (Shan zha/placebo) throughout the seven meeting. Asterisk sign indicates significance level of <0.05. Error bars: +/−1 SD.

FIG. 16b: Graph showing mean changes in score of question 12 of HAM-A between treatment groups (Shan zha/placebo) between time 5 and time 6. Asterisk sign indicates significance level of <0.05. Error bars: +/−1 SD.

FIG. 16c: Graph showing mean changes in score of question 14 of HAM-D between treatment groups (Shan zha/placebo) between time 5 and time 6. Asterisk sign indicates significance level of <0.05. Error bars: +/−1 SD.

FIG. 17: Graph showing mean change in weight (kg) between the treatment group (Shan zha/placebo) throughout the first trial period. Error bars: +/−1 SD.

FIG. 18a-18b: The effect of treatment with various ethanol-based fractions of Shan-zha (10%, 20%, 50% and 70% ethanol) on synaptosomal [³H] 5-HT uptake.

FIG. 18a: is a graph showing %[³H] serotonin uptake inhibition following treatment with Shan-zha's ethanol fractions. Synaptosomes were incubated with the different fractions in the presence of [3H] serotonin and their ability to inhibit its uptake was evaluated (n=2/treatment). Results are presented as mean±SEM.

FIG. 18b: is a graph showing % [3H] 5-HT uptake as a function of the amount (in μg) of each of the 50% and 70% ethanol fractions. Significant negative correlations between the amount of fraction and [3H] 5-HT uptake in vitro were observed for both the 50% ethanol fraction (r=−0.574, P=0.001) and the 70% ethanol fraction (r=−0.441, P=0.021).

FIG. 19a-19b: The effect of treatment with SZ-50 and SZ-70 fractions on anxiety-like behavior.

FIG. 19a: Stressed mice treated with SZ-50 (3 mg/kg, n=20), SZ-50 (30 mg/kg, n=21) and SZ-70 (3 mg/kg, n=10) fractions exhibited reduced anxiety-like behavior in the EPM compared to vehicle-treated stressed mice (n=21) in a similar manner to Shan-zha (30 mg/kg, n=20), NHT (30 mg/kg, n=20) and escitalopram (15 mg/kg, n=20).

FIG. 19b: Stressed mice treated with SZ-50 (3 mg/kg, n=20) and SZ-50 (30 mg/kg, n=21) fractions exhibited reduced anxiety-like behavior in the OFT compared to vehicle-treated stressed mice (n=21) in a similar manner to Shan-zha (n=20), NHT (n=20) and escitalopram-treated mice (n=20).

Results are presented as mean±SEM. **p<0.05. *p<0.001 vs. vehicle; #p<0.05 vs. naïve.

FIG. 20a-20b: The effect of treatment with SZ-50 and SZ-70 fractions on depressive-like behavior and locomotion.

FIG. 20a: Stressed mice treated with SZ-50 (3 mg/kg, n=20), SZ-50 (30 mg/kg, n=21) and SZ-70 (3 mg/kg, n=10) fractions exhibited reduced depressive-like behavior in the TST compared to vehicle-treated stressed mice (n=21) in a similar manner to Shan-zha (30 mg/kg, n=20), NHT (30 mg/kg, n=20) and escitalopram-treated mice (15 mg/kg, n=20).

FIG. 20b: Stressed mice treated with SZ-50 (3 mg/kg, n=20) and SZ-50 (30 mg/kg, n=21) fractions exhibited reduced depressive-like behavior in the FST compared to vehicle-treated stressed mice (n=20) in a similar manner to Shan-zha (n=20), NHT (n=20) and escitalopram-treated mice (n=20). Results are presented as mean±SEM. **p<0.05, *p<0.001 vs. vehicle; #p<0.05, ##p<0.001 vs. naïve.

FIG. 21: The effect of treatment with SZ-50 and SZ-70 fractions on hippocampal BDNF levels.

Significant elevations in hippocampal BDNF levels were observed in mice treated with SZ-50 (3 mg/kg, n=4), SZ-50 (30 mg/kg, n=4) and SZ-70 (3 mg/kg, n=4) fractions compared to vehicle-treated stressed mice in a similar manner to Shan-zha (30 mg/kg, n=3), NHT (30 mg/kg, n=4) and escitalopram-treated mice (15 mg/kg, n=3). Results are presented as mean±SEM. **p<0.05, *p<0.001 vs. vehicle; ##p<0.001 vs. naïve.

FIG. 22: Time (seconds) spent in open arms in the Elevated Plus Maze (EPM).

Effect of treatment on anxiety-like behavior. Mice treated with saline spent significantly less time in open arms in comparison to other treatment groups (Escitalopram, NHT (novel herbal treatment), SZ (Shan-zha), SZ-20% and SZ-50%—Shan-zha fractions extracted in 20% and 50% ethanol). Note: **p<0.01, *p<0.05.

FIG. 23: Forest plot for hazard ratios of sexual activity.

Hazard ratio is calculated for each treatment group (NHT (novel herbal treatment), SZ (Shan-zha), SZ-20% and SZ-50%—Shan-zha fractions extracted in 20% and 50% ethanol) against Escitalopram group. Hazard ration value higher than 1 is considered in favor of the selected treatment over Escitalopram.

### DETAILED DESCRIPTION OF EMBODIMENTS

The present invention is based on studies showing behavioral and biochemical effects of a novel herbal treatment, the Chinese herb Shan-zha, on anxiety and depression symptoms in mouse models.

As will be exemplified in the Examples below, 4-week-old male mice were subjected to unpredictable chronic mild stress (UCMS) for 4 weeks, after which they were treated with various herbs, a combination thereof, the SSRI escitalopram or control (vehicle) for 3 weeks. Anxiety- and depressive-like behaviors as well as brain-derived neurotrophic factor (BDNF) levels in the hippocampus and PFC were assessed. In addition, weight change, sexual function and SERT levels in the PFC were evaluated. Shan-zha was found to concomitantly reduce anxiety- and depressive-like behaviors while increasing BDNF levels in both hippocampus and PFC. Importantly, as opposed to escitalopram, Shan-zha did not change SERT levels and precluded sexual dysfunction and weight gain; side effects associated with reduced adherence to therapy.

These findings suggest that Shan-zha may serve as an efficacious and safe alternative to conventional drugs for treating stress, anxiety, and depression. In addition, as shown below in the Examples, results of clinical studies show that treatment with Sha-zha were safe and had efficient anti-depressive effects, without exhibiting any side effect.

Moreover, ethanol extraction of Shan-zha produced fractions which also showed significant anxiolytic and anti-depressive effects suggesting that these active fractions can also serve as therapeutic agents for treating stress, anxiety, and depression.

Therefore, in one of its aspects, the present invention provides a pharmaceutical or nutritional composition, comprising hawthorn fruit (shan za), or an active fraction extracted thereof, as the active agent, for the treatment or alleviation of symptoms of anxiety disorders, stress, or depression in a subject.

As used herein the term “pharmaceutical composition” refers to a composition comprising hawthorn fruit (shan za), or an active fraction extracted thereof, wherein said composition is provided as a medicament. As used herein the term “nutritional composition” refers to a composition comprising hawthorn fruit (shan za), or an active fraction extracted thereof, wherein said composition is provided as a nutrition additive.

As used herein the term “Shan-Zha” (also termed herein hawthorn plant or Crataegus pinnatifida) refers to dry extract of the plant's fruit.

The term “treatment or alleviation of symptoms” is used conventionally and refers to the management or care of a subject for the purpose of combating, alleviating, reducing, relieving, or improving a subject's anxiety condition, stress, depression, or any symptom thereof. The term encompasses any reduction in the subject's anxiety condition, stress, or depression as evidenced, for example, by a subject's personal report, by suitable questionnaires, or by measurement of physiological indicators of anxiety, stress, or depression, e.g., blood cortisol levels, whereby high levels of cortisol are indicative of stress.

As used herein the term “anxiety disorders” refers to different forms of abnormal and pathological fear and anxiety. The term encompasses anxiety disorders characterized by continuous or episodic symptoms including generalized anxiety, phobic, and panic disorders.

Anxiety disorders are characterized by mental apprehension, and various physical symptoms such as physical tension.

As used herein the term “stress” encompasses both chronic and acute stress conditions.

Symptoms of depression are well known to a person skilled in the art and include, but are not limited to, suicidal tendency.

In one embodiment, the pharmaceutical composition comprises Shan-za dissolved in a salt solution (e.g., saline)+DMSO (dimethyl sulfoxide), preferably in 1% DMSO.

Accordingly, as used herein the term a “Shan-zha fraction” or a “Shan-zha ethanol extract”, refers to fractions of Shan-zha which were obtained by subjecting Shan-zha to various ethanol concentrations, for example, but not limited to 10%, 20%, 50% or 70% ethanol. Specifically, 20%, 50% or 70% ethanol.

The pharmaceutical compositions of the invention can be administered and dosed by the methods of the invention, in accordance with good medical practice, for example the pharmaceutical composition can be introduced to a site by any suitable route including intraperitoneal, subcutaneous, transcutaneous, topical, intramuscular, intraarticular, subconjunctival, or mucosal, e.g., oral, intranasal, or intraocular administration.

More specifically, the compositions used in any of the methods of the invention, described herein, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, formulations are prepared by uniformly and intimately bringing into association the active ingredients of the invention with liquid carriers or finely divided solid carriers or both, and then, if necessary, shaping the product.

The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

In addition to the ingredients particularly mentioned above, the formulations may also include other agents conventional in the art having regard to the type of formulation in question. For example, the compositions of the invention may be incorporated into food products, beverages (e.g., juices) or combined with commonly used food additives such as corn syrup.

Still further, pharmaceutical preparations are compositions that include one or more targeting cassette present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable, or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating, and coloring agents may be used. Pharmaceutical compositions may be formulated into preparations in solid, semisolid, liquid, or gaseous forms, such as tablets, capsules, powders, granules, ointments, solutions, suppositories, injections, inhalants, gels, microspheres, and aerosols.

As such, administration of the composition of the invention can be achieved in various ways, including oral, buccal, rectal, parenteral, intraperitoneal, intradermal, or transdermal administration. The active agent may be administered systemically or may be locally administered. Local administration may be facilitated by using an implant that acts to retain the active dose at the site of implantation. The active agent may be formulated for immediate activity or it may be formulated for sustained release.

Still further, the composition/s of the invention and any components thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the composition of the invention or of any component thereof may last up to a day, two days, three days, four days, five days, six days, a week, two weeks, three weeks, four weeks, a month, two months three months or even more. Specifically, application may last from one day to one month. Most specifically, application may last from one day to 7 days.

In some embodiments the pharmaceutical or nutritional composition of the invention further comprises an additional component (namely an additional active agent other than Shan-zha) which enhances the clinical effect of the composition and generates a beneficial effect in the subject at an early stage of the treatment. For example, after one week, two weeks or three weeks of treatment.

In certain embodiments the additional active agents include, but are not limited to, antioxidants (e.g., selenium), vitamins (such as vitamin A, B1, B2, thiamine, B6, pyridoxine, B complex, biotin, nicotinic acid, B12, C, ascorbic acid, D, D2, D3, E, riboflavin, K, K1 or K2), Co Enzyme Q10, NADH, NAD, D-ribose, or amino acids such as L-Glutamine or Lysine.

The composition of the invention can be administered alone, or in combination with other active agent(s). The compositions of the present invention may be combined with other treatments including behavioral therapy, diet restrictions and pharmacological intervention. Various drugs are known in the art for the treatment of anxiety disorders, stress, or depression, and these can be combined with the compositions of the present invention.

In another aspect, the present invention provides a method of treating or alleviating symptoms of anxiety disorders, stress or depression comprising administering to a patient in need thereof an effective amount of a composition according to the invention, wherein said amounts are effective to treat or alleviate symptoms of anxiety disorders, stress or depression.

By the term “administering”, it is meant that the composition is delivered to a subject by any means or route which is effective to achieve the desired result, including e.g. oral, parenteral, enteral, intraperitoneal, topical, transdermal (e.g. using any standard patch), subcutaneous, intravenous, intra-arterial, intramuscular, buccal, sublingual, ophthalmic, nasal, by aerosol, by inhalation, rectal, vaginal and intrathecal.

In one embodiment the composition of the invention is administered to human subjects in an amount of 1 g/day to about 15 g/day, between about 2 g/day to about 3 g/day, or about 2.5 g/day, or about 10 g/day. In some embodiments the composition of the invention is administered to human subjects in an amount of between about 1 mg/kg to 100 mg/kg, between about 2 mg/kg to 50 mg/kg or between about 3 mg/kg to 30 mg/kg, depending upon the subject's physical condition, the severity of disease, etc. Compositions can be administered at any suitable time, e.g., prior or after a meal, prior to activity, prior to sleeping and at different times of the day, e.g., in the morning, in the evening etc.

### EXAMPLES

### Experimental Procedures of Examples 1-3

Animals

ICR outbred 28-30 days old male mice (Envigo, Israel) were maintained in the vivarium of the Open University lab in Hadassah Ein Karem medical cement, Jerusalem. Mice were group housed (5 mice per cage) and each cage contained mice from all treatment groups. Mice were given ad libitum access to food and water except during stressor application (with the exclusion of the light/dark cycle reversal). Mice kept on a reversed 12 h light/dark cycle (lights on 7:00 pm-7:00 am) while experimental procedures were performed during the dark phase under red light. All experiments were approved by the Open University of Israel committee for animal care and use. Methods were carried out in accordance with the NIH guidelines.

Drugs

Crataegus pinnatifida, Triticum aestivum, Lilium brownii and Fructus zizyphi jujuba were purchased as freeze-dried granules (KPC Products, Inc., Irvine, CA, USA). Each herb was dissolved in saline with 1% dimethylsulfoxide (DMSO) to a final concentration of 0.47 mg/ml and injected i.p. individually (30 mg/kg) or with the other components to form the formula (NHT, 30 mg/kg). Escitalopram was dissolved in saline (+1% DMSO) and injected i.p. (15 mg/kg). Vehicle treatment included i.p. injection of saline with 1% DMSO. All treatments were carried out for 3 weeks.

Elevated Plus Maze (EPM)

The EPM task was performed as previously described [6]. Shortly, each mouse was placed in the center of the maze and video recorded for 5 min. Anxiety-like behavior was expressed as the time the mouse spent in the open, unprotected arms of the maze.

Tail Suspension Test (TST)

The TST task was performed as previously described [7]. Shortly, mice were suspended from a horizontal bar by taping the tip of their tail to the bar for 6 min, and the time spent in immobile positions during the last 4 min was evaluated.

Open Field Test (OFT)

The open field consists of an empty square arena (40×40×40 cm) and surrounded by Perspex opaque walls. Each mouse was placed in the center of the arena and video recorded for 5 min. Task performance was later coded using the Biobserve software (BIOBSERVE GmbH, Augustin, Germany). The arena was thoroughly cleaned with ethanol and allowed to dry between subjects in order to eliminate any odor cues. Locomotor activity was expressed as the percentage of time that the mouse was moving in the arena in a velocity above 0.1 pixel/sec.

Body Weight Monitoring

Mice were weighed every 3 days throughout the treatment period. Change in weight was calculated as final (weight-initial weight)/initial weight×100.

Evaluation of Sexual Function

Each male mouse was placed with a female mouse in estrus in the male's home cage during the dark phase under red dim light for 30 min. Number of mounts were coded by an observer blind to the treatment.

High Affinity [³H] Citalopram Binding Assay

PFC samples were disrupted with Brinkman polytron in 50 vol of buffer (50 mM Tris-HCl, 120 mM NaCl and 5 mM KCl at a pH of 7.4) and centrifuged (×3) at 30,000×g for 10 min. The pellet was resuspended in the same buffer to yield a final concentration of approximately 21 mg/ml (wet weight). [³H] citalopram binding was determined by a standard binding assay that contained 100 μl of brain homogenate, 100 μl [3H] citalopram (0.54 nM) and 300 μl buffer. After a 60 min incubation period at 25° C., homogenates were diluted in 3 ml ice-cold buffer and filtered with vacuum through Whatman GF/C glass fiber filters. Filters were washed (×3) with 3 ml ice-cold buffer, and radioactivity was measured in scintillation liquid using a β-counter (Packard, Tri-Carb 2100TR). Specific binding was defined as the difference between total [³H] citalopram binding (triplicate samples) and non-specific binding in the presence of 10 μM fluoxetine (duplicate samples). Protein concentration was measured by the method of Lowry et al (Willner, et al. Neurosci Biobehav Rev, 1992. 16(4): p. 525-34).

### STATISTICAL ANALYSIS

Data were analyzed using one-way ANOVA with treatment as a between subject variable. ANOVA was followed by the following post-hoc tests: Dunnett for EPM, TST and BDNF data; LSD for SERT, weight change and sexual function data. Significance was assumed at p<0.05.

### Example 1

Treatment with Shan-Zha Reduced Anxiety- and Depressive-Like Behaviors in Stressed Mice

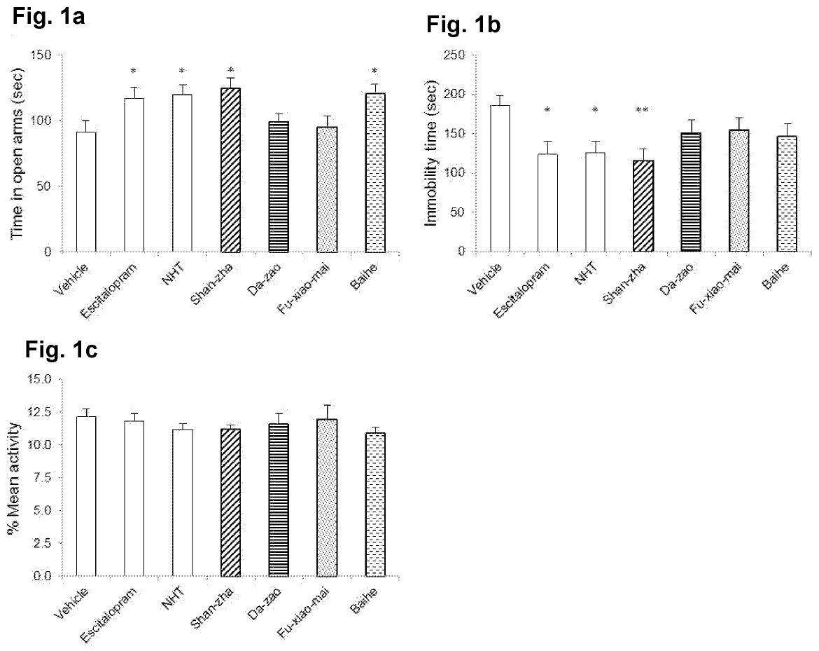

One-way ANOVA revealed a significant effect of treatment on anxiety-like behavior in the EPM (F(_{6.98})=3.121, p<0.008). Shan-zha- and Baihe-treated mice spent more time in the open arms of the maze in comparison with vehicle-treated mice (post-hoc p<0.008, p<0.022, respectively) in a similar manner to escitalopram- and NHT-treated mice (p<0.049 and p<0.023, respectively) (FIG. 1A). Escitalopram is one of the most commonly used drugs for the treatment of anxiety in Israel. Contrast analysis between Shan-zha- and Baihe revealed a non-significant difference (p<0.740). One-way ANOVA revealed a significant effect of treatment on depressive-like behavior in the TST (F(_{6.93})=3.121, p<0.046). Shan-zha-treated mice spent less time immobile in comparison with vehicle-treated mice (p<0.005) in a similar manner to escitalopram- and NHT-treated mice (p<0.021 and p<0.020, respectively) (FIG. 1B). No differences in locomotion were observed in OFT suggests there was no motor impairment to confound findings in the EPM and TST analysis, and that none of the treatments induced sedative or stimulant effects (F(6,98)=0.605, p<0.726) (FIG. 1C).

### Example 2

Treatment with Shan-Zha Increased BDNF Levels in the Hippocampus and PFC of Stressed Mice

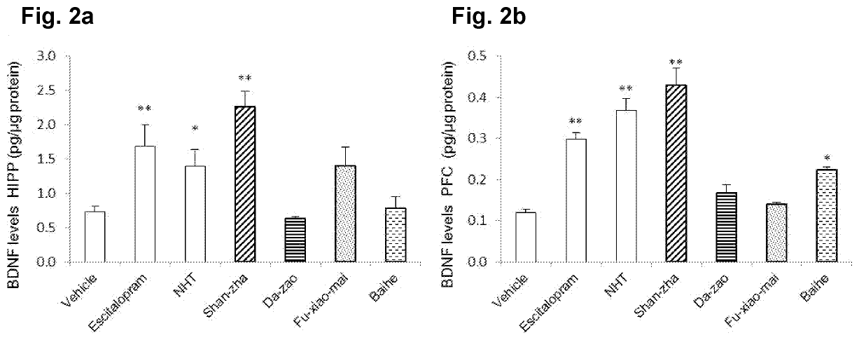

One-way ANOVA revealed a significant effect of treatment on hippocampal BDNF levels (F(_{6.35})=7.09, p<0.001). Shan-zha-treated mice exhibited elevations in hippocampal BDNF levels compared to vehicle-treated mice (p<0.001) in a similar manner to escitalopram- and NHT-treated mice (p<0.004, p<0.048, respectively) (FIG. 2A). One-way ANOVA revealed a significant effect of treatment on BDNF levels in the PFC (F(_{6.28})=26.34, p<0.001). Shan-zha and Baihe-treated mice exhibited elevations in BDNF levels in the PFC compared to vehicle-treated mice (p<0.001 and p<0.011, respectively) in a similar manner to escitalopram- and NHT-treated mice (p<0.001, p<0.0001, respectively). Contrast analysis between Shan-zha- and Baihe revealed a significant difference with Shan-zha having higher levels of BDNF in the PFC (p<0.001) (FIG. 2B).

### Example 3

Treatment with Shan-Zha Did not Reduce SERT Levels in the PFC and Did not Cause Weight Gain and Sexual Dysfunction in Stressed Mice

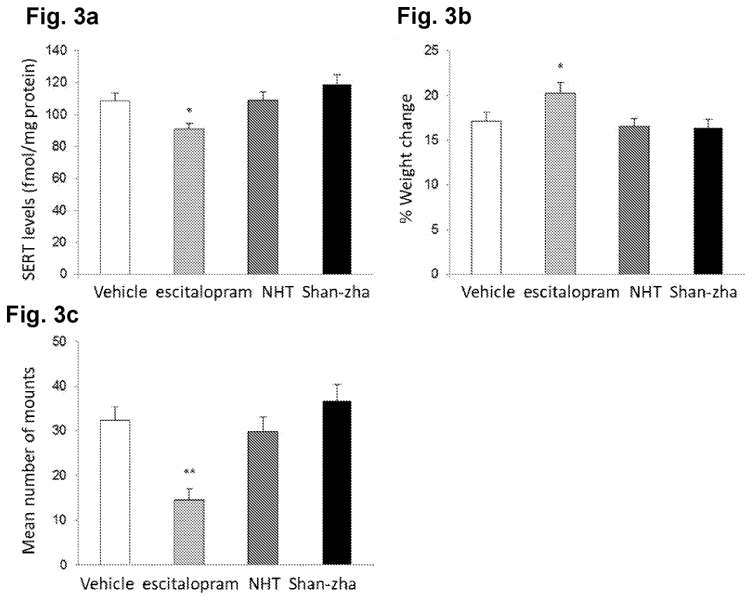

One-way ANOVA revealed a significant effect of treatment on SERT levels in the PFC (F(_{3.33})=4.81, p<0.007). Shan-zha-treated mice exhibited comparable SERT levels with NHT- and vehicle-treated mice (p<0.168 and p<0.161, respectively). Escitalopram-treated mice exhibited lower SERT levels in comparison with vehicle-treated mice (post-hoc p<0.033) (FIG. 3A). One-way ANOVA revealed a significant effect of treatment on weight change (F(_{3.71})=3.174, p<0.029). Shan-zha-treated mice exhibited a comparable weight gain with vehicle-treated mice (p<0.582), similarly to NHT-treated mice (p<0.684). Escitalopram-treated mice exhibited higher weight gain in comparison with vehicle-treated mice (p<0.036) (FIG. 3B). One-way ANOVA revealed a significant effect of treatment on sexual function (F(_{3.74})=8.902, p<0.001). Shan-zha-treated mice exhibited comparable number of mounts vehicle-treated mice (p<0.323), similarly to NHT-treated mice (p<0.581). Escitalopram-treated mice exhibited lower number of mounts in comparison with vehicle-treated mice (p<0.001) (FIG. 3C).

### Experimental Procedures of Examples 4-5

Animals

Female (n=40) and male (n=5) ICR outbred mice (100 days old; Envigo RMS, Jerusalem, Israel) were housed at 22±1° C. under 12 h light/dark cycles in the vivarium of the Open University lab in Hadassah medical center, Jerusalem. Mice had ad libitum access to rodent chow and water. Mice were housed in standard cages with a bed of woodchips and a piece of cotton wool for enrichment. All experiments were performed during the dark phase of the cycle. Male mice were used to father pups and were housed apart (5 per cage) from females until mating. Female mice (3 per cage) had a week of acclimation and coordination of the estrus cycle in the home cage and then assigned for mating cage (2 females and 1 male per cage). Once a vaginal plug was identified, the females were transferred to a separate cage until parturition. Overall, 455 pups were delivered of which 317 (male: n=165; female: n=152) were designated for this study. Unassessed pups were maintained with littermates to avoid additional stress and ensure similar conditions. Similar amounts of mice were utilized per litter to avoid litter effects. All experiments were approved by the Open University of Israel Committee for Animal Care and Use. Methods were carried out to minimize animal suffering in accordance with NIH guidelines.

Pharmacological Agents

On postnatal day (PND) 1, dams were randomly assigned to three treatment groups: escitalopram (15 mg/kg), shan-zha (15 mg/kg) or control. Drugs were administered daily via dorsum subcutaneous injection (to mitigate possible damage to mammary glands that could be caused by intraperitoneal injection) until PND21, when the period of rapid brain growth in mice is due to cease (Johansson N, et al (2009) Toxicol Sci 108:412-418). Escitalopram was kindly donated by Teva Ltd. (Petah-Tikva, Israel)). Shan-zha was purchased from KPC Products (CA, USA) as freeze-dried granules. Drugs were dissolved in 1% DMSO saline. Controls were injected with the vehicle. Doses were opted based on previous studies [5-6].

Stressor

On gestational day (GD) 15, dams were randomly assigned to stress (n=20) or control (n=20) group. Dams in the stress group were individually restrained daily in transparent plastic cylinders (30 mm diameter) under bright light (650 lux) three times a day, for 45 min until parturition, as previously described (Van Den Hove DLA, et al (2005) Dev Neurosci 27:313-320). Control dams were kept undisturbed in their home cages during gestation.

Biochemical Assessments

Following behavioral assessments on PND21 dams and pups, blood samples were obtained from the facial vein into EDTA-coated tubes. Shortly after mice were subjected to cervical dislocation and their brains were placed on dry ice. The hippocampus was dissected out entirely and immediately stored (−80° C.) for later analysis.

BDNF Enzyme-Linked Immunosorbent Assay (ELISA)

Mice were decapitated, and their brains were placed on ice. Serial sections (1 mm wide) were cut onto slides, and tissue punches of the hippocampus and PFC (1.7 mm diameter) were taken. Tissue punches were homogenized in cold extraction buffer (Tris-buffered saline, pH 8.0, with 1% NP-40, 10% glycerol, 5 mM sodium metavanadate, 10 mM PMSF, 100 μg/ml aprotinin and 10 μg/ml leupeptin). Homogenates were acidified with 0.1 M HCl (pH 3.0), incubated at room temperature (22-24° C.) for 15 min, and neutralized (pH 7.6) with 0.1 M NaOH. Homogenates were then microfuged at 7000×g for 10 min. BDNF levels were quantified using sandwich ELISA (R&D systems, Minneapolis, MN, USA) according to the manufacturer's instructions.

Blood Serum [³H]Citalopram Binding Assay

Blood samples were centrifuged (6,000×g; 4° C.) for 10 min and serum was separated. To examine the presence of escitalopram in the serum high affinity [3H]citalopram binding assay was used. A different set of naïve mice were decapitated and their brains were dissected on ice. The frontal cortex of the mice were disrupted with Brinkman polytron in 50 vol of ice-cold buffer (50 mM Tris-HCl, 120 mM NaCl and 5 mM KCl; pH 7.4) and centrifuged (30,000×g) for 10 min (×3). The pellet was resuspended in the same buffer to yield a final concentration of 30 mg/ml (wet weight). [³H]citalopram binding was determined by a standard binding assay that contained 50 μl of brain homogenate, 50 μl [³H]citalopram (0.5 nM) and 150 μl buffer. Serum assays were determined in the presence of 25 ul of the examined serum. After a 60 min incubation period at room temperature, the samples were washed with 3 ml ice-cooled buffer (×3) and filtered with vacuum through Whatman Glass microfiber filters GF/C (GE Healthcare Life Sciences, IL, USA). The radioactivity was measured in Tri-Carb 2100TR liquid scintillation counter (Packard) using a β-counter. Specific binding was defined as the difference between total [³H]citalopram binding and the non-specific binding in the presence of 10 μM fluvoxamine.

Essentially, the [³H]citalopram binding assay examined the percentage of serotonin transporters (SERT) that was free to bind radioactive ligands in the homogenate, as measured by β-counter. This percentage is an inverse measure of serum escitalopram concentration, since higher concentration of escitalopram in the serum would result in more escitalopram from the serum binding to SERT in the homogenate, resulting in a lower concentration of free SERT that could bind to the radioactive ligand. Thus, low concentration of free SERT would indicate high concentration of escitalopram in the original serum sample.

Study Design

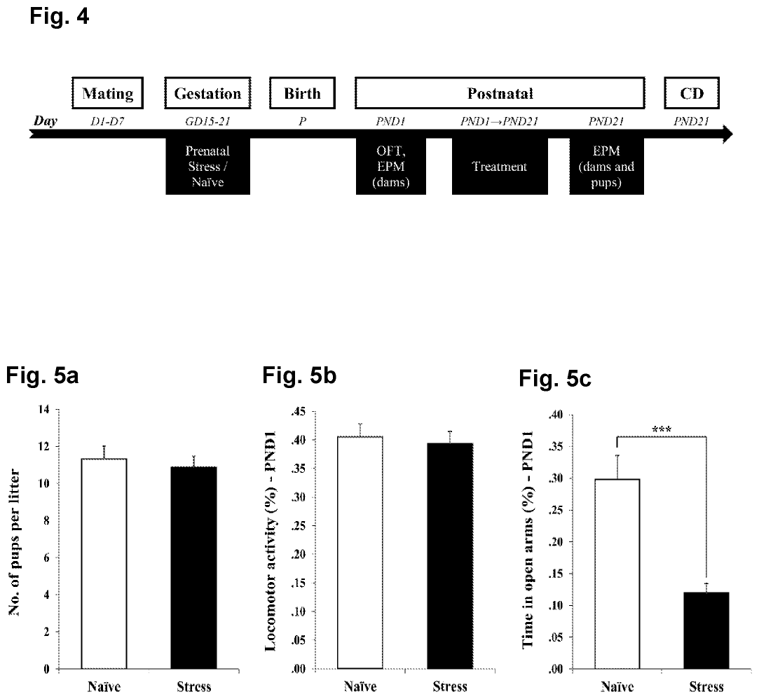

Following mating, dams were separated to individual cages and were observed periodically to ensure onset of pregnancy. On GD15 dams were randomly assigned to manipulation group (stress/naïve). Dams' number of pups per litter and locomotor activity in the OFT (on PND1) were obtained to negate cardinal physiological deficit as a confounding explanation. Shortly after, anxiety-like behavior was assessed on the EPM and dams were assigned to treatment group (escitalopram/shan-zha/vehicle). Treatments were administered for three weeks (PND1→PND21). On PND21 dams and pups were subjected to the EPM and immediately thereafter prepared for biochemical assessments (see FIG. 4 for study design).

Unpredictable Chronic Mild Stress (UCMS)

The procedure was performed during adolescence, starting at the age of 4 weeks. Individually housed mice were exposed to UCMS as previously described [7] for 4 weeks, 4 hours a day, using the following stressors: restraint, placement in an empty cage with water at the bottom, switching cages, cage tilting, wet sawdust and reversal of the light/dark cycle. To prevent habituation and to provide an unpredictable feature to the stressors, the order of stressor application was counterbalanced across subjects and treatment groups.

### Example 4

Effects of Shang-Zha on Dams

4.1 Locomotor Activity on PND1 and Litter Size—Stress During Gestation had No Effect on Dams' Locomotor Activity and Litter Size

Independent samples t-tests did not reveal a significant difference between stressed and naïve dams on number of pups delivered per litter (N.S.; FIG. 5A) or on locomotor activity in the OFT on PND1 (N.S.; FIG. 5B) (OFT was conducted as described for Examples 1-3 above). This suggests that stress manipulation did not cause imminent physical impairment to dams.

4.2 Anxiety-Like Behavior—Shan-Zha and Escitalopram Normalized Long-Term Anxiety-Like Behavior Induced by Stress During Gestation in Dams

EPM was conducted as described above for Examples 1-3. On PND1, independent samples t-test revealed a significant difference between the groups in time spent in the open arms in the EPM (t(₂₈)=4.44, p<0.001; FIG. 5C). On the day following parturition, stressed dams demonstrated increased anxiety-like behavior compared to naïve dams.

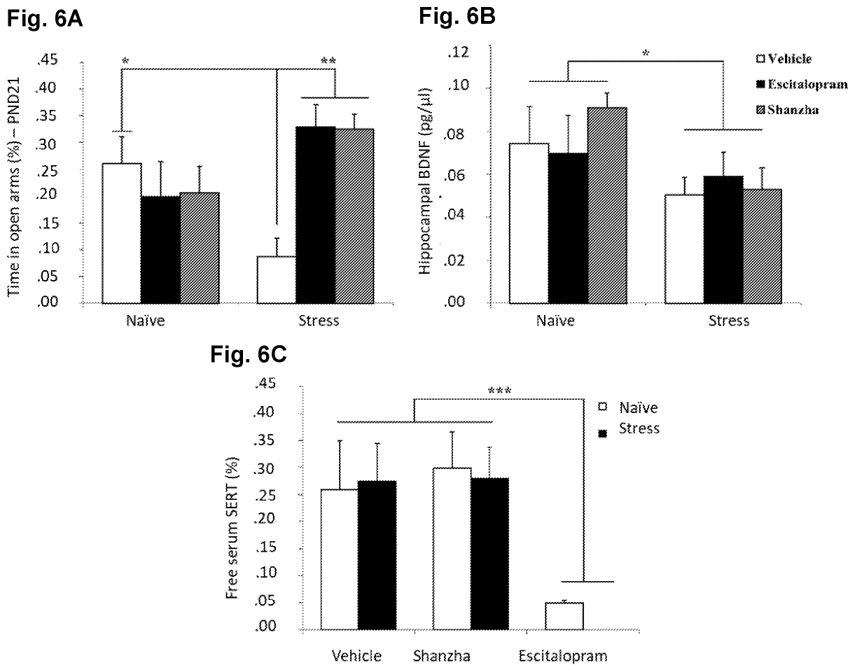

On PND21, two-way ANOVA [stress×treatment (2×3); FIG. 6A] on anxiety-like behavior in the EPM revealed a significant stress×treatment interaction (F(_{2.24})=6.96, p<0.01), with no significant main effects for stress (N.S.) or treatment (N.S.). Analysis of simple effects revealed that saline-treated stressed dams spent less time in the open arms of the maze (i.e., increased anxiety-like behavior), compared to saline-treated naïve dams (p<0.05). Stress manipulation did not affect time spent in open arms in both the shan-zha and escitalopram groups (N.S.). Moreover, saline-treated stressed dams demonstrated elevated anxiety-like behavior compared to both shan-zha- and escitalopram-treated stressed dams (p<0.01 in both contrasts). No difference between the treatment groups was observed under the naïve condition (N.S.).

4.3. Hippocampal BDNF Concentration—Stress During Gestation Reduced Dams' Hippocampal BDNF Concentration 21 Days Post-Parturition

Two-way ANOVA [stress×treatment (2×3); FIG. 6B] on hippocampal BDNF concentration revealed a significant effect for stress (F(_{1.17})=5.81, p<0.05), with no significant interaction and treatment effects (N.S.). Dams undergone restrain stress during gestation had lower concentration of BDNF in the hippocampus 3 weeks post-parturition, regardless of pharmacological treatment.

4.4. Serum SERT Binding—Increased Amount of Escitalopram was Found in Escitalopram-Treated Dams

Two-way ANOVA [stress×treatment (2×3); FIG. 6C] on percentage of SERT free for binding in dams' serum revealed significant effect for treatment (F(_{2.18})=14.61, p<0.001), with no significant interaction and stress effects (NS.). Dunnett post-hoc analysis revealed that the escitalopram group had significantly less SERT free for binding compared to both the saline and the shan-zha groups (p<0.001 in both contrasts). This indicates that dams that were treated with escitalopram had higher concentration of escitalopram in their serum compared to the other groups (the escitalopram in the serum bound with the SERT molecules, and the radioactive ligand had less free SERT to bind to).

### Example 5

Effects of Shan-Zha on Pups

To assess sex differences three independent samples t-tests were conducted on anxiety-like behavior in the EPM [males: X̅=0.287 (0.014); females: X̅=0.275 (±0.015)], hippocampal BDNF concentration [males: X̅=0.073 (+0.003); females: X̅=0.072 (±0.004)] and free serum SERT levels [males: X̅=0.315 (±0.018); females: X̅=0.318 (±0.022)]. In all three tests no significant differences were observed between male and female pups (NS.).

5.1. Anxiety-Like Behavior—Shan-Zha and Escitalopram Via Lactation Normalized Prenatal Stress-Induced Anxiety-Like Behavior in Pups

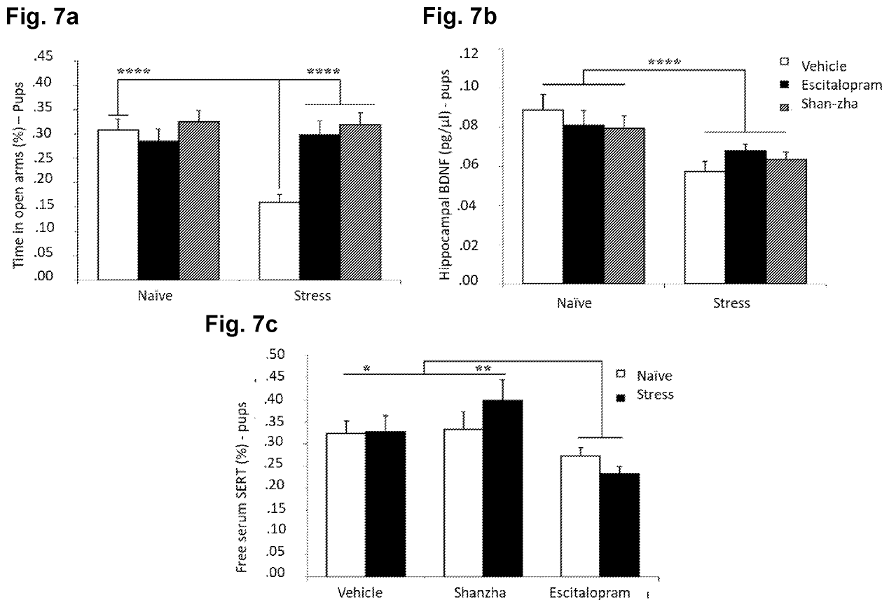

On PND21, two-way ANOVA [stress×treatment (2×3); FIG. 7A] on anxiety-like behavior in the EPM revealed significant effects for treatment (F(_{2.215})=7.2, p<0.001), stress (F(_{1.215})=5.8, p<0.05) and stress×treatment interaction (F(_{2.215})=6.91, p<0.001). Analysis of simple effects revealed that saline-treated stressed pups spent less time in the open arms, compared to saline-treated naïve pups (p<0.0001). No similar stress-induced anxiety-like behavior was observed in the shan-zha and escitalopram groups (N.S.). Moreover, saline-treated stressed pups demonstrated elevated anxiety-like behavior compared to both shan-zha- and escitalopram-treated stressed pups (p<0.0001 in both contrasts). No difference between the treatment groups was observed under the naïve condition (N.S.).

5.2. Hippocampal BDNF Concentration—Prenatal Stress Induced a Reduction in Pups' Hippocampal BDNF Concentration

Two-way ANOVA [stress× treatment (2×3); FIG. 7B] on pups' hippocampal BDNF concentration revealed a significant effect for stress (F(_{1.82})=17.25, p<0.0001), with no significant interaction and treatment effects (N.S.). Prenatal stress yielded a reduction in BDNF levels in the hippocampus of 21 days old pups, regardless of treatment (via lactation) group.

5.3. Serum SERT Binding—Increased Amount of Escitalopram was Found in Pups of Escitalopram-Treated Dams

Two-way ANOVA [stress× treatment (2×3); FIG. 7C] on percentage of SERT free for binding in pups' serum revealed a significant effect for treatment (F(_{2.84})=5.6, p<0.01), with no significant interaction and stress effects (N.S.). Dunnett post-hoc analysis revealed that the escitalopram group had significantly less SERT free for binding compared to both the saline group (p<0.05) and the shan-zha group (p<0.01). This indicates that pups that were nursed by dams treated with escitalopram had higher concentration of escitalopram in their serum compared to the other treatment groups. Hence, escitalopram was excreted into dams' milk and increased serum escitalopram concentration of offspring via lactation.

Evaluation of Alterations in Gene Expression Related to Side Effects Differentially Induced by SZ and Escitalopram.

An Unbiased genome-wide transcriptomic profiling was conducted to reveal alterations in gene expression in the hippocampus induced by treatment. Male ICR mice were subjected to UCMS, after which they were treated with Shan-zha (30 mg/kg), NHT (30 mg/kg), Esciatlopram (15 mg/kg) or saline for 3 weeks. The anxiolytic-like effect of the treatments was verified by EPM. Subsequently, brains were rapidly removed and RNA was isolated from the hippocampus using RNeasy kit (Qiagen). RNA-sequencing (on NGS) and gene expression analysis was conducted. The analysis yielded a wide range of changes in gene expression.

First, all samples were normalized and clustered in order to verify that there are no noticeable biases based on batches/process/technician/day.. Next, hit maps were generated, in order to observe the global differences between the groups. After which, a PCA analysis was performed to visualize the separation between the samples. Finally, Differential expression analysis with DESE-Q was done, and changes in gene expression of the following genes, which are highly relevant to anxiety, depression or drug-related side effects were followed:

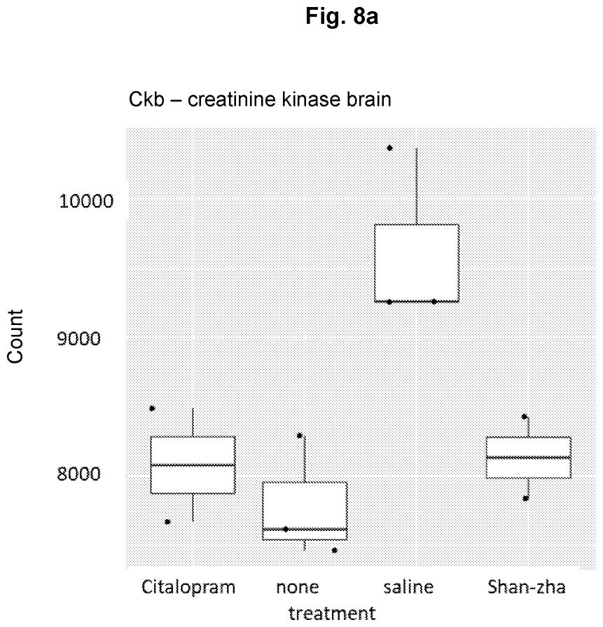

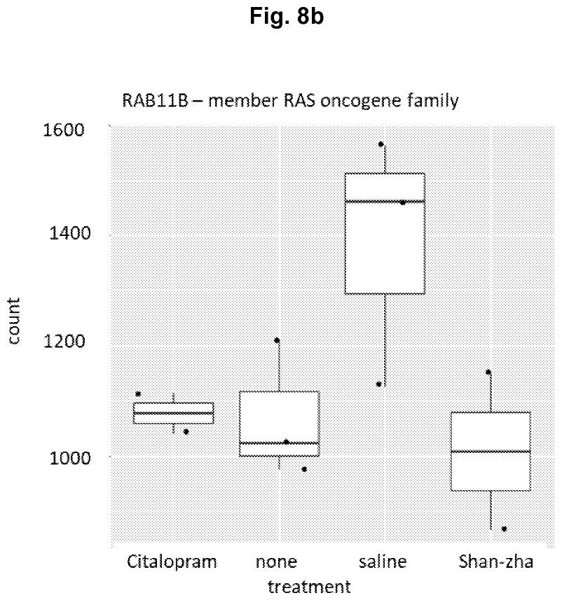

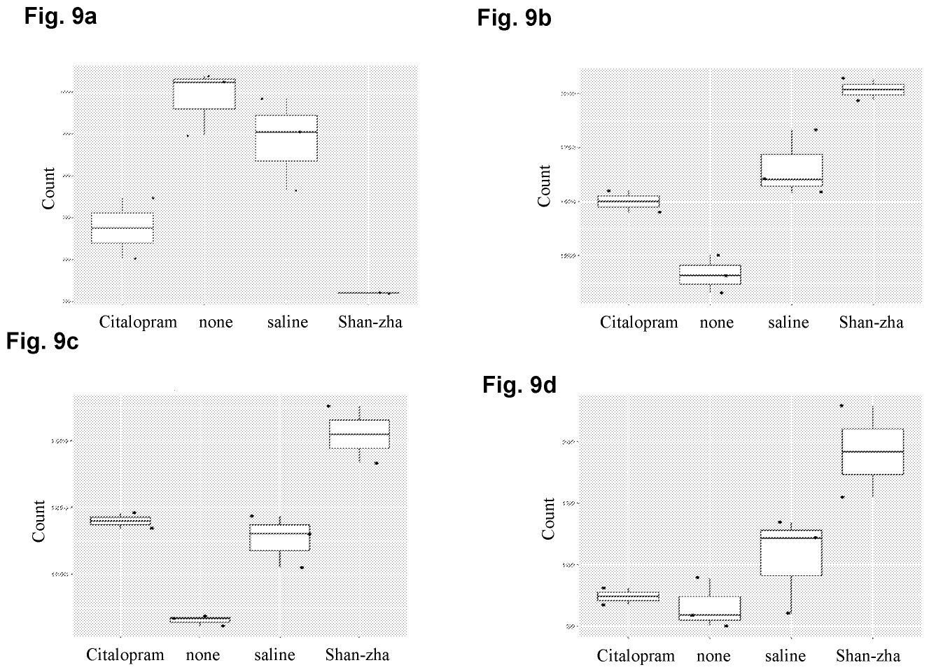

Creatine kinase B-type (CKB), encoding for the cytoplasmic enzyme that is involved in energy homeostasis; and RAB11 b, a key regulator of intracellular membrane trafficking. Both are also known to be elevated in depression. These genes were found to be downregulated in both Shan-zha and ecitalopram treatment groups (CBk, fold change of 0.84 and 0.83; RAB11b, fold change of 0.72 and 0.77, respectively. P<0.05), compared to the UCMS-exposed saline treated group (FIG. 8a and FIG. 8b). In contrast, the IGSF9B gene is significantly under expressed in the Shan-zha group (fold change of 0.58, p<0.01) and is known to be implicated in schizophrenia and bipolar disorder. It also has a role in the promotion of inhibitory synapse development, and it has been shown to be upregulated during anxiety, while its deletion inhibits anxiety-like behavior in mice (FIG. 9a).

Interestingly, some genes were specifically upregulated in the Shan-zha group and not in the ecitalopram (FIG. 9b-d), these genes were related to mechanisms that upregulate BDNF and serotonin levels (Mif, with fold change of 1.21), mitochondria functions (Ndufa3, with fold change of 1.34) and suppress weight gain (Hint2, with fold change of 1.8). These may be responsible in some part to the lack of known side-effects in the Shan-zha —treated group compared to the Citalopram-treated group.

### Example 6

Clinical Trials with Shang-Zha

The study included two research approaches. First, a double blind, randomized cross over trial was performed to elucidate the therapeutic potential of Shan-zha as a novel treatment for anxiety disorders. Secondly, a variety of molecular and biochemical methods were used to validate and elucidate the molecular mechanism that underlies the effectiveness of the Shan-zha as a new pharmacological approach for treating anxiety.

6.1. To Evaluate the Therapeutic Efficacy of the Novel Herbal Treatment (Shan-Zha) on Anxiety Symptoms Relative to SSRI:

The effect of Shan-zha treatment on improvement of anxiety symptoms was clinically studied. Individuals with anxiety disorders or persons with anxiety characterization were treated with either Shan-zha, or SSRI treatment.

Experimental Design: This arm was done at the Mazor Mental Health Center in Akko, Israel and affiliated outpatient clinics. Subjects: Overall 50 individuals with anxiety disorders were enrolled from Mazor outpatients' clinics.

Inclusion criteria: Inclusion criteria for all participants were as follows:
1) Subjects meeting the DSM-V criteria for one or more of the following: Generalized Anxiety Disorder (GAD), Panic Disorder and Social Anxiety Disorder;
2) Males and females;
3) Age 18-60 years;
4) Not receiving antidepressant drugs for at least two weeks;
5) Hamilton Anxiety Rating Scale (HAM-A) scores>17 and <30.

Exclusion criteria: Exclusion criteria for all participants were as follows:
1) Current active and persistent substance and/or alcohol abuse;
2) Current or past history of delusion or hallucination;
3) Past history of at least one manic episode, hypomanic episode, or mixed episode;
4) Mental retardation;
5) Systolic Blood Pressure <100 mm Hg;
6) Heart rate <60 times/min;
7) HAM-A score >30;
8) Psychotherapy within 6 weeks prior to study entry.

Criteria to terminate the trial: Trial termination criteria for all participants were as follows:
1) Worsening of anxiety or/and depressive symptoms;
2) Changes in the Patient's general health conditions;
3) Developing suicidal thoughts;
4) Adverse side effects.

Study design: A 12-week randomized, double-blind, cross-over study was performed. After providing written informed consent of their willingness to participate in research, subjects (In total, n=50) were randomized to receive treatment with either Shan-zha (2.5 gr); 1-2 capsules three times a day) or the SSRI escitalopram (10 mg) daily for 6 weeks, followed by a blind crossover for another 6 weeks. In addition, subjects were asked to fill a socio-demographic questionnaire, and to undergo a clinical differential diagnosis using the Symptoms Check List (SCL)-90 and Clinical Global Impression (CGI).

The SCL-90 was used to obtain a more detailed characterization of symptoms. The questionnaire evaluates a broad range of psychological problems and symptoms of psychopathology, and measures nine primary symptom dimensions. The questionnaire is also designed to provide an overview of a patient's symptoms and their intensity at a specific point in time.

CGI is a brief assessment tool in psychiatry that measures illness severity (on a scale from 1 (normal) to 7 (severe)), global improvement or change (on a scale from 1 to 7), and therapeutic response (on a scale from 0 (improvement) to 4 (unchanged or worse)). The CGI is developed for use in NIMH-sponsored clinical trials to provide a brief, stand-alone assessment of the clinician's view of the patient's global functioning prior to and after initiating a study medication.

Anxiety and depression symptoms were evaluated using the Hamilton anxiety rating scale (HAM-A) and Hamilton depression rating scale (HAM-D), respectively. The HAM-A was developed to rate the subject's severity of anxiety before, during, and after treatment. It is based on the clinician's interview with the subject and probes both psychic anxiety (mental agitation and psychological distress) and somatic anxiety (physical complaints related to anxiety). While, HAM-D was developed to rate the patient's level of depression before, during, and after treatment. It is based on the clinician's interview with the patient and probes symptoms such as depressed mood, guilt feelings, suicide, sleep disturbances, anxiety levels, and weight loss.

The Sheehan Disability Scale (SDS) was used to measure the subject's quality of life. The questionnaire is designed to measure the level of three major sectors in the patient's life (work, family, and social life) affected by anxiety and depressive symptoms. It rates the extent to which the patient's work, social life, or leisure activities, and home life or family responsibilities are impaired by his or her symptoms on a 10-point visual analog scale. The scale may be used as a self-report, administered by a clinician, or rated by both independently.

Adverse effects of the treatment was measured by the Treatment Emergent Symptom Scale. Clinical improvement was monitored every two weeks till the end of the trial using HAM-A, HAM-D, CGI, and SDS.

Study outcomes: The primary outcome measures was the improvement of anxiety and depressive symptoms, while the secondary outcome was subjective and objective wellbeing. HAM-A and HAM-D were used to monitor treatment progress, whereas SDS was used to monitor the subject's functional improvement in work, social, and family life.

Specific Methodology:

Drugs: Shan-zha, has been approved as a food supplement by the State of Israel Ministry of Health. The component was purchased as freeze-dried granules from KPC Products, Inc., and was capsulated in 0.5 gram capsule (manufacture by Bara Herbs, Yokne'am, Israel). Escitalopram was purchased from Lundbeck Israel LTD.

Statistical Analysis: ANOVA was used to analyze continuous demographic and adverse side effects variables between the groups. Categorical variables, including incidence of adverse events was analyzed using Chi-square (λ²) test. ANOVA analysis of variance was used to monitor treatment progress and subjects' functional improvement, followed by multiple comparisons. Data was analyzed using the SPSS 21.0 statistical analysis software package.

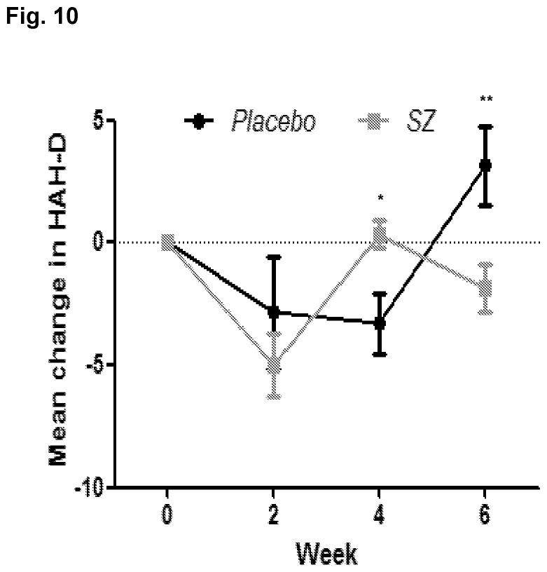

The results of the study are summarized in Table 1. FIG. 10 shows the specific effect of Shan zha on depression symptoms versus placebo. It therefore appears that the treatment with Shan zha attenuates anxiety-like behavior, moderates the hormonal reaction to acute and chronic stress, increases neurogenesis and has minor side effect.

TABLE 1
Demographic and clinical data.

| | Placebo (n = 8) | SZ (n = 16) |
|---|---|---|
| Demographic | | |
| Age (years) | 39.6 ± 3.11 | 38.4 ± 2.9 |
| Education (years) | 12.4 ± 0.7 | 12.0 ± 0.5 |
| Gender (%) | Men - 89% | Men - 100% |
| | Women - 11% | Women - |
| Clinical | | |
| Years of illness | 12.8 ± 3.1 | 10.6 ± 2.7 |
| \hat{ }Short Zung | 53.4 ± 2.1 | 53.3 ± 1.6 |
| \hat{ }HAM-D | 10.1 ± 1.7 | 11.1 ± 1.5 |
| \hat{ }PANSS - positive | 13.6 ± 1.9 | 11.7 ± 0.9 |
| \hat{ }PANSS - negative | 15.3 ± 2.3 | 14.9 ± 1.7 |
| \hat{ }CGI - severity | 3.1 ± 0.2 | 3.4 ± 0.1 |

Results represent mean ± SEM;
\hat{ }Score at week 0.

6.2. To Study the Molecular Target of the Novel Herbal Treatment (Shan-Zha):

The molecular mechanism underpinning the therapeutic efficacy of Shan-zha is studied using a variety of molecular and biochemical methods. Specifically, the effect of the treatment on neurotropic factors, pro-inflammatory cytokines and gene expression profile is evaluated in the blood samples of the subjects.

Experimental Design:

6.3. The Effect of the Shan-Zha Herb on Mild to Moderate Depression and Anxiety

This study included 67 individuals, 21 males and 46 females, suffering from mild to moderate symptoms of depression and/or anxiety based on the Hamilton Depression Rating Scale (HAM-D) and the Hamilton Anxiety Rating Scale (HAM-A). Participants were recruited to the study through advertisements in various forms of media, such as Facebook, television shows, and the Open University's website. Overall, 1,340 individuals applied to participate in the study. Applicants were asked to fill out an on-line questionnaire, intended to test their compatibility to the study (see inclusion and exclusion criteria). Final compatibility to the study based on depression and/or anxiety symptoms assessed using the HAM-D and the HAM-A, along with evaluation of the reliability of the answers given on the on-line questionnaire, was determined during an introductory assessment meeting. Assessment was completed by M.A clinical psychology students with the assistance of psychiatrists from “Mazor” Hospital in Acco.

Inclusion Criteria:
Hamilton Depression Rating Scale (HAM-D) scores of >8 and <18 and/or Hamilton Anxiety Rating Scale (HAM-A) scores of >17 and <30
Age 18-65
Nonconsumption of antidepressants/anxiolytic drugs for at least three months prior to onset of trial

Exclusion Criteria:
Current active substance and/or alcohol abuse
Mental retardation
Other major psychiatric disorders: Psychosis, Severe MDD, and Bipolar Disorder.
Suicide ideation
Pregnancy
Systolic Blood Pressure <100 mmHg
Heart rate <60 times/min
Medical history of cardiac palpitation and other cardiac diseases

Termination Criteria:
Worsening of depressive and or anxiety symptoms
Changes in general health conditions
Developing suicidal thoughts
Adverse side effects

Table 2 presents demographic data categorized by treatment group (Shan-Zha/placebo). As presented, most subjects were female (68%), between the ages of 26-50, and single (68%). Approximately half of the subjects had high-school level education while the other half had higher level education.

TABLE 2

| | All | | | | | | p- |
|---|---|---|---|---|---|---|---|
| | subjects (60) | | Shan-Zha (37) | | Placebo (23) | | value |
| Age | 37.69 | (11.94) | 37.43 | (12.76) | 38.09 | (10.75) | 0.84 |
| Gender | | | | | | | 0.46 |
| Male | 19 | (32%) | 13 | (35%) | 6 | (26%) | |
| Female | 41 | (68%) | 24 | (65%) | 17 | (74%) | |
| Marital | | | | | | | 0.26 |
| Status | | | | | | | |
| Single | 41 | (68%) | 26 | (70%) | 15 | (65%) | |
| Married | 13 | (22%) | 6 | (16%) | 7 | (30%) | |
| Divorced | 6 | (10%) | 5 | (14%) | 1 | (5%) | |
| Education | | | | | | | 0.60 |
| High-School | 26 | (46%) | 17 | (56%) | 9 | (39%) | |
| B.A/M.A | 34 | (54%) | 20 | (44%) | 14 | (61%) | |

Demographic Data Categorized by Treatment Group

Continuous variables are represented by the mean and the standard deviation; categorical variables are represented by frequencies and percentages. Between groups comparisons were executed using T-Test, Chi-Square tests and Fisher's exact test. This data excludes participants who dropped out following initial assessment.

Tools

Demographics Questionnaire: Compromised of variables such as: age, gender, family status, country of birth, first language, spoken languages, parents' nationality, years of education, military service (yes/no) and other non-psychiatric illnesses.

Hamilton Depression Rating Scale (HAM-D) (Hamilton, 1960): A semi-structured interview developed to rate the subject's level of depression before, during, and after treatment. The scale consists of 21 items that probe symptoms such as depressed mood, feelings of guilt, suicide, sleep disturbances, anxiety levels, and weight loss. Question 14 assesses sexual drive. Each item is scored on a scale of 0-4 or 0-2 so that higher scores imply higher severity of symptoms. The total score is calculated by adding all item scores. For the purposes of this study, only items 1-17 were included in the calculation of the total score. Items 17-21 are supplementary items, added to the original 17 items for the intention of classifying depression type. The internal reliability of the original scale is good (α=0.84) (Hamilton, 1960). However, the internal reliability of the scale throughout the seven measurements of the study was questionable (α=0.60).

Hamilton Anxiety Rating Scale (HAM-A) (Hamilton, 1959): A semi-structured interview developed to rate the subject's level of anxiety before, during, and after treatment. The scale consists of 14 items that probe both psychic anxiety (mental agitation and psychological distress) and somatic anxiety (physical complaints related to anxiety). Question 12 assesses sexual functioning. Each item is scored on a scale of 0 (not present) to 4 (severe). The total score is calculated by adding all item scores. The internal reliability of the original scale is adequate (α=0.78) (Steer, 1987). Correspondingly, the internal reliability of the scale throughout the seven measurements of the study was adequate (α=0.73).

The Sheehan Disability Scale (SDS) (Sheehan, 1983): Developed in order to rate the subject's quality of life. The scale is designed to measure the quality level of three major sectors of life (work, social life, and home life) affected by anxiety and depression. It rates the extent to which the patient's work, social life or leisure activities, and home life or family responsibilities are impaired by his or her symptoms on a 10-points visual analog scale. This visual analog scale uses numeric and verbal descriptive anchors simultaneously to assess disability. The SDS has previously demonstrated adequate internal consistency with Cronbach's a ranging from 0.56-0.89 (Arbuckle et al., 2007; Hodgins, 2013; Leon et al., 1992; Leon et al., 1997). For the purposes of this study, the scale was used as a self-report. The internal reliability of the scale throughout the seven measurements of the study was excellent (α=0.86).

Visual Analogue Scales (VAS): A measurement instrument for subjective characteristics or attitudes that cannot be directly measured. The scale is administered as a horizontal line, 107 mm in length, anchored by word descriptors at each end. The subject marks on the line the point which they feel represents his/her current state. For the purposes of this study, two such continuous scales were used; not at all anxious (left end) to extremely anxious (right end) and depressed mood (left end) to good mood (right end). With respect to anxiety, greater values indicate a more severe current state, as opposed to mood for which lower values indicate a more severe current state. The VAS score is determined by measuring (in millimeters) the distance from the left end of the line to the point marked by the subject (Aitken, 1969).

Clinical Global Impression (CGI) (Guy, 1976): Developed for use in NIMH-sponsored clinical trials to provide a brief, stand-alone assessment of the clinician's view of the patient's global functioning prior to and after initiating medication. The CGI measures illness severity on a scale from 1 (normal) to 7 (severe) and global improvement or change from baseline on a scale from 1 (normal) to 7 (severe). Global improvement or change is reported as of the second meeting with the subject.

Assessment of Adverse Effects: A checklist of all known adverse effects following treatment with Shan-Zha.

Study Design

The study was approved by the Helsinki committee of the “Mazor” hospital (permission no. 0007-17-MZR). A 6-weeks, randomized, double-blind, placebo-controlled trial was performed. Following the initial trial period, a 6-weeks open-label extension phase was implemented. Individuals who were found suitable to participate in the study via the online questionnaire and an initial assessment meeting signed an informed consent form. Overall, 67 subjects were recruited to the study; 44 were enrolled to the treatment arm and 23 to the placebo arm.

Following acceptance to the study, subjects were randomized to Shan-Zha (2.5 grams. 5 capsules a day; 3 in the morning and 2 in the evening) or placebo treatment (identical in substance quantity and consumption instructions) in a 2:1 ratio, respectively. Shan-Zha was purchased as freeze-dried granules from KPC products, Inc., and was capsulated in 0.5 grams capsules (Manufactured by Bara Herbs, Yokne'am, Israel). Starch served as placebo and was capsulated in 0.5 grams capsules (Manufactured by Bara Herbs, Yokne'am, Israel).

During the introductory assessment meeting, subjects who were accepted to the study completed the demographics questionnaire, the SDS, and the VAS, and CGI scores were obtained from clinicians. Baseline scores of clinical assessments categorized by treatment group are presented in table 3. As presented, most subjects received a Hamilton-D score in the range of 9-17 and a Hamilton-A score in the range of 11-24. SDS scores in all three domains (work, social, and family) were mostly in the range of 3-8. VAS mood scores were mostly in range of 2-7, and VAS anxiety scores were mostly in the range of 3-9. CGI scores mostly ranged from 3-5.

TABLE 3
Baseline clinical data categorized by
treatment group subjects

| | All | | | | | | p- |
|---|---|---|---|---|---|---|---|
| | subjects (60) | | Shan-Zha (37) | | Placebo (23) | | value |
| Hamilton | 12.84 | (3.92) | 13.08 | (4.19) | 12.46 | (3.48) | 0.55 |
| Depression | | | | | | | |
| Hamilton | 17.43 | (6.60) | 18.08 | (7.50) | 16.37 | (4.78) | 0.33 |
| SDS - Work | 5.36 | (2.09) | 5.30 | (1.84) | 5.45 | (2.46) | 0.81 |
| SDS - Social | 5.93 | (1.84) | 5.83 | (1.75) | 6.09 | (2.00) | 0.61 |
| SDS - Family | 5.51 | (2.10) | 5.61 | (2.18) | 5.35 | (2.01) | 0.64 |
| VAS Mood | 4.16 | (2.38) | 4.16 | (2.59) | 4.21 | (2.05) | 0.89 |
| VAS Anxiety | 6.03 | (2.87) | 5.94 | (3.03) | 6.18 | (2.66) | 0.76 |
| CGI | 3.82 | (1.27) | 3.78 | (1.47) | 3.87 | (0.87) | 0.80 |

All variables are represented by the mean and the standard deviation. Between groups comparisons were executed using T-tests. This data excludes participants who dropped out following initial assessment.

Following the baseline assessment, subsequent assessment meetings were held every 2 weeks for a period of 12 weeks; a total of 7 assessment meetings were held. In each meeting, clinicians assessed depression and anxiety symptoms severity through the HAM-D, the HAM-A, and the CGI, and subjects reported their subjective states through the SDS and the VAS. In addition, subjects' physiological factors (pulse, blood pressure, and weight) were obtained and an assessment of adverse effects was completed.

Meetings 1-4 constituted the double-blind trial period. At the end of the double-blind trial period, all subjects, regardless of treatment group, were immediately enrolled into a 6-week open-label extension phase. In this phase, all subjects received Shan-Zha treatment identical to the one given during the first trial period; subjects who were randomized to the placebo group started to knowingly receive the Shan-Zha and subjects who were randomized to the Shan-Zha group kept receiving the Shan-Zha. Subjects were aware of this change but were not aware of the group to which they were initially randomized (Shan-Zha/placebo). During the extension phase, the time period between the 5^{th} and 6^{th} meetings was of particular interest to the study. During this time, 2-4 weeks after the beginning of the extension phase, it was expected that the Shan-Zha to begin taking effect among subjects who were previously randomized to the placebo group while continuing to affect subjects who have been receiving Shan-Zha throughout the initial trial period, but to a lesser degree.

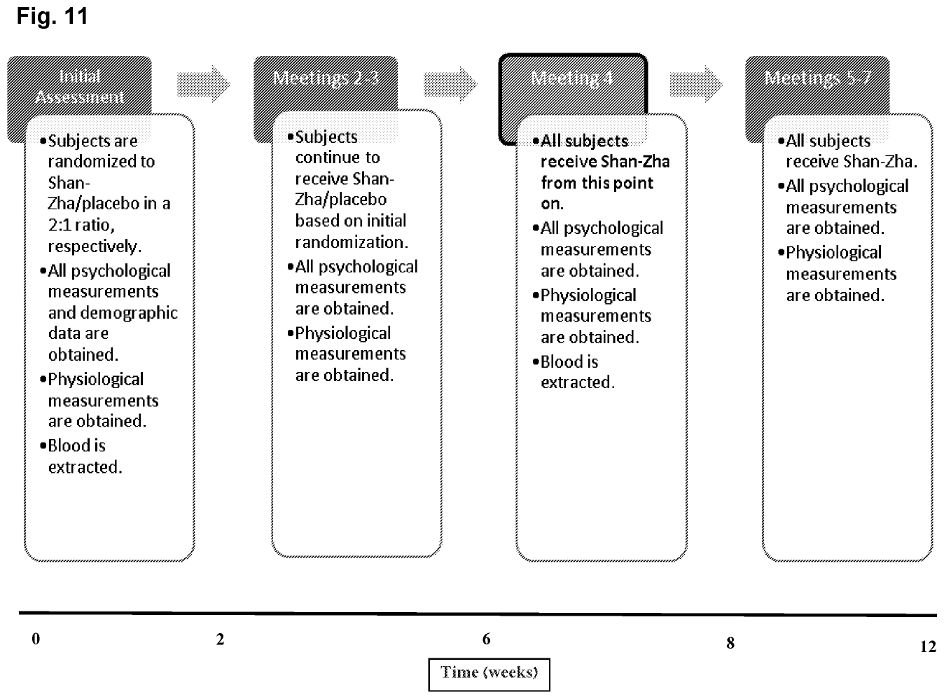

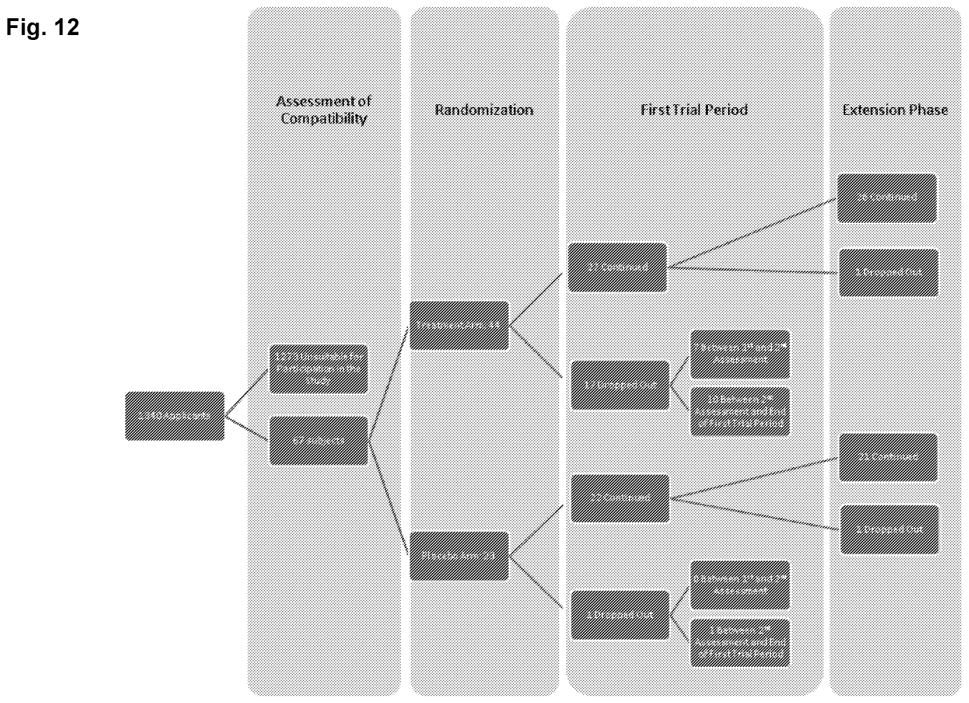

During the 1^{st} and 4^{th} meetings, blood samples were extracted from subjects in order to analyze changes in blood composition following consumption of Shan-Zha. Throughout the study period, continuous daily reports of subjective states were obtained through an Immediate Mood Scaler (IMS) application installed on the subjects' phones. Other than the assessment of weight change, the physiological aspects of the study, as well as the technological approach, will be discussed further in separate papers. The timeline of the study is presented in FIG. 11.

As illustrated in FIG. 12, out of 1340 applicants, 67 individuals were accepted to the study. A total of 20 subjects dropped out of the study throughout the 12 week-period. Reasons for dropout were various and unrelated (e.g., schedule overload and the onset of psychiatric treatments).

In order to test the hypotheses, both remission rates and change in symptom severity were analyzed. When analyzing remission rates, the difference in the percentage of subjects who reaches remission (received a Hamilton Depression Rating Scale score of less than 8) was first analyzed by the end of the first trial period between the two treatment groups (Shan-Zha/placebo). Second, out of all subjects who reached remission during the first trial period, the difference in the time (measured by meeting number) it took subjects to reach remission between the treatment groups was analyzed. When analyzing change in symptom severity, mean differences in scores of all measurements were examined. Each analysis included subjects for whom all measurements (across time) were available. Reasons for missing values were various and unrelated.

Remission (yes/no) Rates and Times

In order to analyze remission rates, “remission” was defined dichotomously based on a Hamilton Depression Rating Scale (HAM-D) cutoff score of 8; subjects who received a HAM-D score of less than 8 were defined as “in remission” and subjects who received aHAM-D score greater than 8 were defined as “not in remission”. No differences were found in the percentage of subjects who reached remission by the end of the first trial period between the treatment groups, λ² (1,N=60)=0.07, p=0.79.

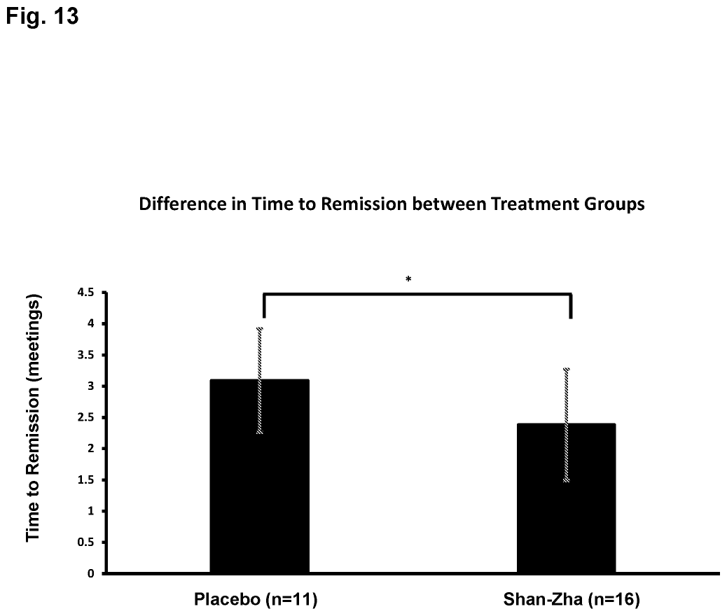

Out of all subjects who reached remission by the end of the first trial period, the difference in the time (measured by meeting number) it took subjects to reach remission between the treatment groups was examined using an independent T-test with treatment group (Shan-Zha, placebo) as the grouping factor and time in which the subject reached remission (time 1, time 2, time 3, time 4) as the dependent variable. In line with our hypothesis, the mean difference in time to remission between the Shan-Zha group (M=2.38, SD=0.89) and the placebo group (M=3.09, SD=0.83) reached statistical significance, t(25)=−2.12, p=0.045. On average, subjects in the Shan-Zha group reached remission quicker than subjects in the placebo group (See FIG. 13).

Change in Symptom Severity

In order to analyze change in symptoms severity of all measurements, several mixed Analyses of Variance (ANOVA) were performed, with treatment group as the between subjects variable and time as the within subjects variable. The dependent variable, as well as the exact time period assessed, varied across analyses according to the hypothesis. Two time periods were of interest:
1) time 1-4, constituting the initial, double-blind trial period.
2) time 5-6, in which it was hypothesized hypothesized that the Shan-Zha would begin to take effect among subjects who were previously randomized to the placebo group while continuing to affect subjects who were previously randomized to the Shan-Zha group, but to a lesser degree. Question 12 of the HAM-A and question 14 of the HAM-D, which assess sexual functioning and sexual drive respectively, were analyzed as stand-alone questions.

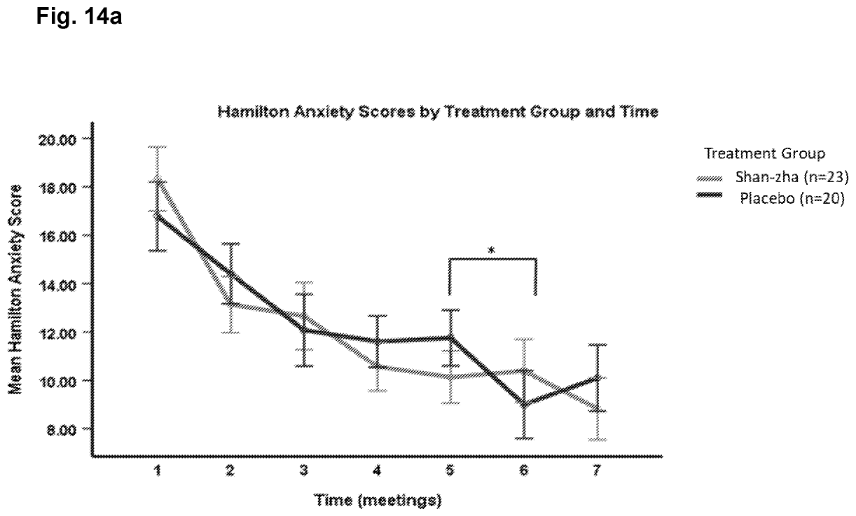

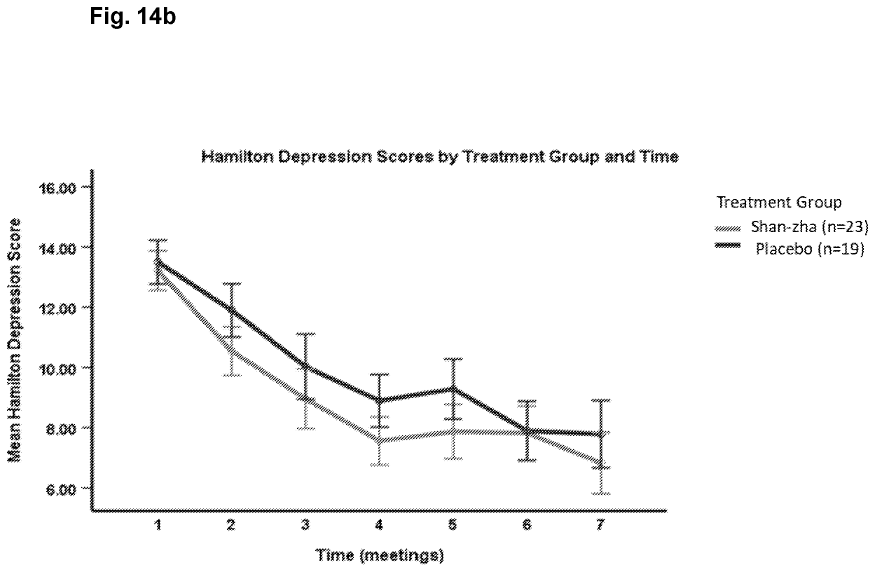

In line with our hypothesis, under the HAM-A, the interaction between treatment group (Shan-Zha, placebo) and time (time 5, time 6) reached statistical significance, F(1,41)=4.26, p=0.045, so that the difference in HAM-A scores for the placebo group between time 5(M=11.75, SD=4.18) and time 6(M=9.00, SD=4.82) reached statistical significance (mean difference=2.75, p=0.01), while the difference in HAM-A scores for the Shan-Zha group between time 5(M=10.13, SD=5.88) and time 6(M=10.39, SD=7.24) did not reach statistical significance (mean difference=−0.26, p=0.80). Between the 5^{th} and 6^{th} meetings, subjects who were previously randomized to the placebo group improved significantly more in HAM-A scores than subjects who were previously randomized to the Shan-Zha group (see FIG. 14A). Under the HAM-D, the interaction between treatment group (Shan-Zha, placebo) and time (time 5, time 6) did not reach statistical significance, F(1,40)=1.04, p=0.31. (See FIG. 14B).

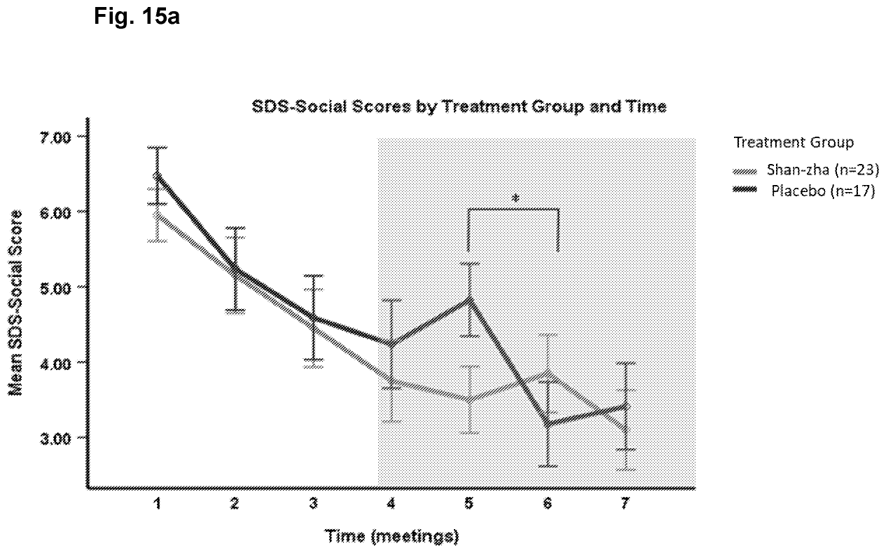

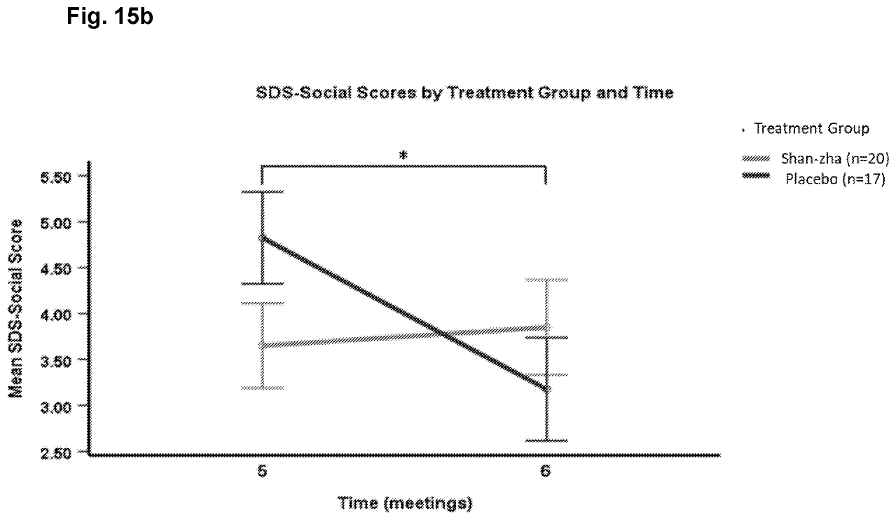

In line with the hypothesis, under the Sheehan Disability Scale (SDS)-Social, the interaction between treatment group (Shan-Zha, placebo) and time (time 5, time 6) reached statistical significance, F(1,35)=4.92, p=0.03, so that the difference in SDS-Social scores for the placebo group between time 5(M=4.82, SD=1.94) and time 6(M=3.18, SD=2.19) reached statistical significance (mean difference=1.65, p=0.01), while the difference in SDS-Social scores for the Shan-Zha group between time 5(M=3.50, SD=2.01) and time 6(M=3.85, SD=2.41), did not reach statistical significance (mean difference=−0.35, p=0.52). Between the 5^{th} and 6^{th} meetings, subjects who were previously randomized to the placebo group improved significantly more in SDS-Social scores than subjects who were previously randomized to the Shan-Zha group. see FIG. 15A-15B.

No differences in SDS family, Visual Analogue Scales (mood and anxiety), and Clinical Global Impression scores between the Shan-Zha group and the placebo group were found between the 5^{th} and 6^{th} meetings. See table 4. SDS-work scores were not analyzed due to missing values.

TABLE 4
Mean differences in SDS (family), VAS (mood, anxiety) and CGI
scores between time 5 and time 6 between treatment groups

| | SDS-F | VAS-M | VAS-A | CGI |
|---|---|---|---|---|
| Shan-Zha | −0.7 | 0.45 | 0.27 | 0.00 |
| Placebo | 0.47 | −0.29 | 0.83 | 0.41 |
| p-value | 0.14 | 0.37 | 0.54 | 0.34 |

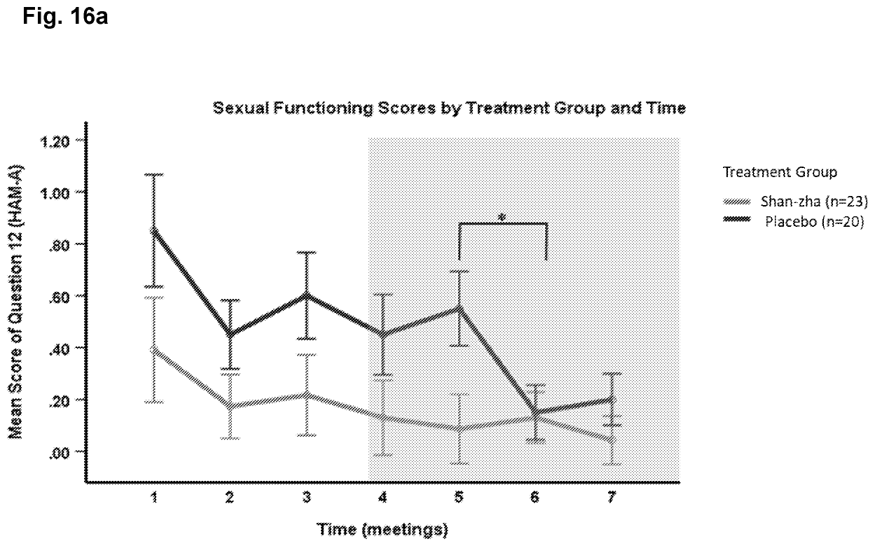

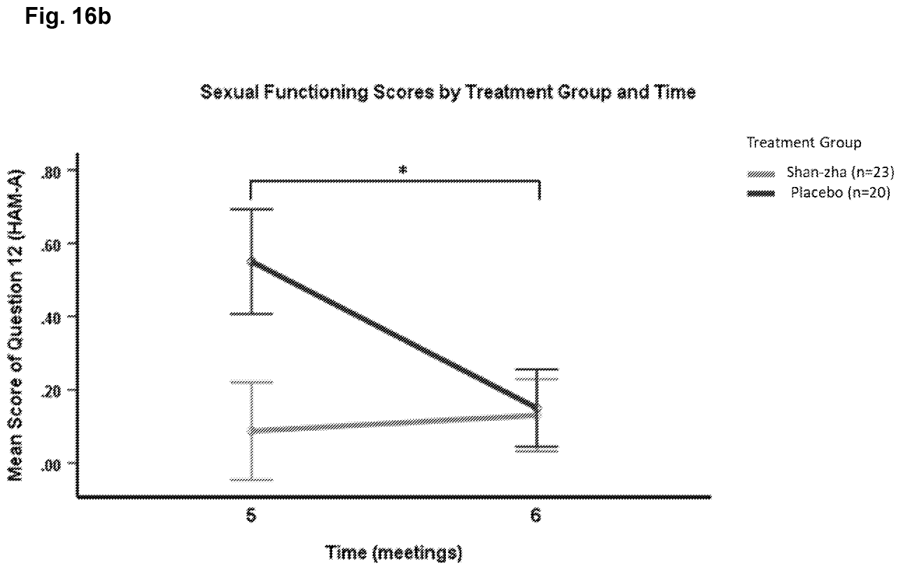

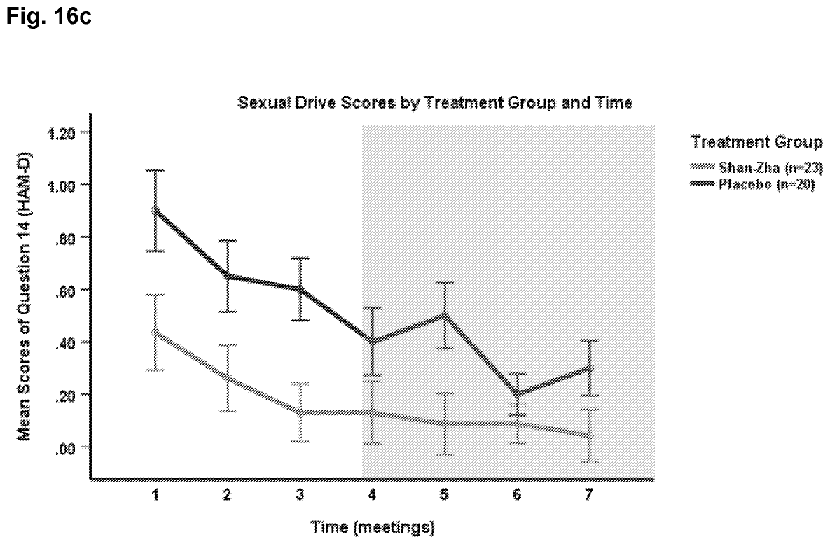

In line with the hypothesis, under question 12 (of the HAM-A), the interaction between treatment group (Shan-Zha, placebo) and time (time 5, time 6) reached statistical significance, F(1,41)=4.36, p=0.04, so that the difference in scores of question 12 for the placebo group between time 5(M=0.56, SD=0.89) and time 6(M=0.15, SD=0.49) reached statistical significance (mean difference=0.4, p=0.01), while the difference in scores of question 12 for the Shan-Zha group between time 5(M=0.09, SD=0.29) and time 6(M=0.13, SD=0.46) did not reach statistical significance (mean difference=−0.43, p=0.76). Between the 5^{th} and 6^{th} meetings, subjects who were previously randomized to the placebo group improved significantly more in scores of question 12 than subjects who were previously randomized to the Shan-Zha group. See FIG. 16A-16B. Under question 14 (of the HAM-D), the interaction between treatment group (Shan-Zha, placebo) and time (time 5, time 6) did not reach statistical significance, F(1,41)=2.78, p=0.10, See FIG. 16C.

No differences between the Shan-Zha group and the placebo group were found throughout the initial, double-blind trial period in all measurements.

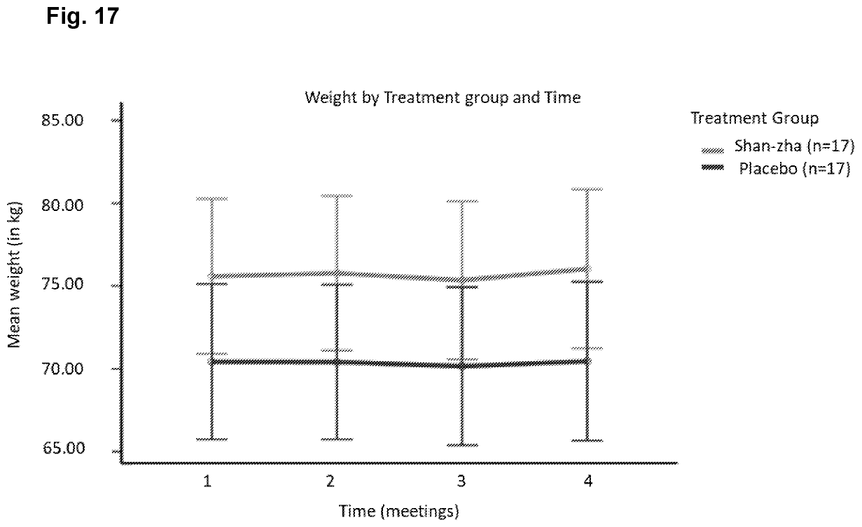

No differences in weight were found between the Shan-Zha group and the placebo group over time, F(3,96)=0.26, p=0.84. See FIG. 17. Weight change was not analyzed throughout the extension phase due to missing values. The subjects completed daily questionnaires. No other adverse effects were reported.

### Experimental Procedures of Example 7

Animals. ICR outbred mice (Envigo RMS (Harlen), Israel) are kept in the vivarium of the Open University lab in Hadassah medical center, Jerusalem. Mice are kept on a reversed 12 h light/dark cycle and given ad libitum access to food and water. All experiments are performed during the dark phase under red light (7:00-19:00). All experiments have been approved by the committee for animal care and use according to the NIH guidelines.

Drugs. Drugs are dissolved in saline and 1% DMSO to the desired concentration and administered i.p. Herbal mixture and Shan-zha (30 mg/kg) will be prepared by dissolving each component (KPC Products, Inc., CA, USA) to a final concentration of 0.47 mg/ml. The SZ-20, SZ-50 and SZ-70 fractions are administered at a daily dose of 30 mg/kg (as the NHT dose), or 3 mg/kg (one tenth of NHT's dose assuming that the fractions should be more potent). Escitalopram is administered at a dose of 15 mg/kg.

Solid phase extraction. Shan-zha are extracted by ethanol and separated using the Clean™ SPE 900 mg Prevail™ C18 fractionating column in HPLC. One gr of Shan-zha plant powder are placed in 20 ml of 20% ethanol and homogenized using POLYTRON® PT 10-35 for 30 sec. The ethanolic mixture are stirred for 30 min on a Fried Electric MH-4 hotplate magnetic stirrer and then are stirred for additional 30 min at 50° C. Finally, the ethanol extract are centrifuged in a UniCen MR centrifuge at 7500 RPM for 10 min. The supernatant are applied to the C18 column (Clean™ SPE 900 mg Prevail™ C18) using the Rocker 300 vacuum system. The column are eluted with distilled water and then with increasing concentrations of ethanol (10, 20, 50, 70 and 100%). The different ethanol fractions are placed in a round glass flask and evaporated via a Vacuum Controller V-850 evaporator followed by freeze-drying phase in a ScanVac CoolSafe freeze dryer. A light brown powder is obtained.

Unpredictable chronic mild stress (UCMS). Mice are exposed to UCMS for 4 weeks at the age of 4 weeks as previously described [7].

Elevated plus maze (EPM). This task is based on the natural tendency of mice to avoid open and elevated places. EPM are performed as previously described [6]. Anxiety-like behavior are expressed as the time the animal spent in the open, unprotected arms of the maze.

Open Field Test (OFT). This test reflects the conflict between the innate fear that mice have of the central area of the field versus their desire to explore new environments. When anxious, the natural tendency of mice is to prefer staying close to the walls. OFT are performed as previously described [5]. Anxiety-like behavior is expressed as the time the animal spent in the center area of the field (Walsh, R. N. and R. A. Cummins, Psychol Bull, 1976. 83(3): p. 482-504). Locomotor activity are expressed as the percentage of time that the mouse moved in the arena in a velocity above 0.1 pixel/sec.

Forced Swim Test (FST). This test is based upon the evaluation of immobility as a measure of behavioral despair and are performed as previously described [7]. Depressive-like behavior are expressed as the time the mouse was immobile defined as cessation of limb movements except minor movement necessary to keep the mouse afloat.

Tail Suspension Test (TST). This test is based upon the evaluation of immobility as a measure of behavioral despair and are performed as previously described [7]. Depressive-like behavior are expressed as the time the mouse was immobile.

Monitoring body weight. Weight is monitored every 3 days during UCMS procedure and treatment.

Evaluation of sexual function. Male mice are placed with a female mouse in estrus, in the male's home cage during the dark phase under red dim light for the duration of 30 min. Male sexual behavior, including number of mounts with or without intromission are recorded.

Assessment of [³H] 5-HT uptake inhibition. The uptake of [3H] 5-HT to mouse brain synaptosomes is performed as previously described. Briefly, the uptake assay will contain: 50 μl synaptosoms supernatant, 50 μl tritiated serotonin and 0.9 ml buffer A (119 mM NaCl, 3.9 mM KCl, 0.65 mM MgSO4, 0.51 mM CaCl2), 0.19 mM sodium phosphate buffer, pH 7.4). The tubes are pre-incubated at 37° C. for 10 min and radioactive serotonin are added (1.0×10-8 M-5×10-7) in 37° C. for 4 min. Specific uptake is defined as the difference between total [3H] 5-HT uptake and non-specific uptake.

Assessment of [³H] citalopram binding inhibition. SERT competitive interaction with [3H]citalopram is assessed as previously described. Shortly, membranes supernatant are incubated with 1 nM [3H] citalopram in the presence of the different fractions for 60 min at 22° C. in a final volume of 250 μl. Specific binding is determined as the difference between total [3H] citalopram binding and the binding in the presence of 10 μM fluoxetine.

Evaluation of SERT levels. SERT levels are evaluated using high affinity [3H] citalopram binding assays as previously described [7]. Shortly, the assay contain 100 μl of brain supernatant, 100 μl [³H] citalopram (0.5 nM) and 300 μl buffer. Specific binding is defined as the difference between total [³H] citalopram binding and the binding in the presence of 10 μM fluoxetine.

Assessment of brain BDNF levels. Tissues are homogenized in cold extraction buffer (Tris-buffered saline, pH 8.0, with 1% NP-40, 10% glycerol, 5 mM sodium metavanadate, 10 mM PMSF, 100 mg/ml aprotinin and 10 mg/ml leupeptin). Homogenates are acidified with 0.1 M HCl (pH 3.0), incubated at room temperature (22-24° C.) for 15 min, and neutralized with 0.1 M NaOH (pH 7.6). Homogenates are then microfuged at 7,000 g for 10 min. BDNF levels in the supernatant are evaluated using sandwich ELISA.

RNA extraction. Total RNA in bloods and brains are isolated using RNeasy Mini Kit (Qiagen) according to the manufacturer's protocol. RNA quality is checked using RNAse free, 1% agarose gel and is quantified using a NanoDrop spectrophotometer (ND-1000).

Gene and miRNA microarray. Affymetrix GeneChip mouse Gene 2.0 ST arrays and Affymetrix GeneChip miRNA 4.0 arrays are used for gene and miRNA expression analysis, respectively, according to the instruction manuals (Affymetrix, Santa Clara, CA, USA). Microarray analysis are performed on CEL files using Partek Genomics Suite™ (Partek, St Louis, MO, USA). Data analysis is performed as previously described.

Real-time PCR. cDNA preparation and real-time PCR experiments are conducted as previously described. Comparative critical threshold (Ct) values obtained by real-time PCR analysis are used for relative quantification of genes or miRNAs expression and determination of fold-change of expression.

Target gene down-regulation. miRNA expressing lentivirus is prepared as previously described. A synthetic miRNA (hsa-miR, Pre-miR; Ambion, Austin, TX) is used for down-regulation. An amount of 1 μl of miRNA expressing lentivirus or a negative control (contains random sequence pre-miR; Ambion, Austin, TX) is stereotaxically injected (N=80) bilaterally into the dentate gyrus (coordinates: AP=−3.0 mm, ML=+2.0 mm, DV=−2.5) or prelimbic cortex (coordinates AP=+2.1 mm, ML=±0.25 mm, DV=−1.5 mm). Streotaxic injections are performed as previously described. Following surgery, mice are housed individually and allowed 4 weeks to recover until the lentivirus will infect most of the cells in the injection site.

BrdU injections. Mice are injected i.p. with BrdU (100 mg/kg, Sigma) once a day for 3 days starting on the first day of treatment.

Statistical analysis. Data is analyzed using one-way ANOVA with treatment as a between subject variable, followed by a post hoc Dunnett or LSD analysis. Significance will be assumed as p<0.05.

### Example 7

Isolation of the Active Ingredient in a Novel Herbal Treatment for Depression and Anxiety: Behavioral and Neuronal Effects in a Mice Model

In order to identify an active ingredient in Shan-zha, ethanol fractions were produced utilizing C18 fractionating column in high-performance liquid chromatography (HPLC).

The specificity of the preparation of the fractions was in the fact that the C18 column was entirely loaded. In common protocols, the C18 column should be loaded only with half the quantity of materials. A column of only 1 gram was used and ethanol fractions of 10%, 20%, 50%, 70% and 100% were produced, dried and finally frozen.

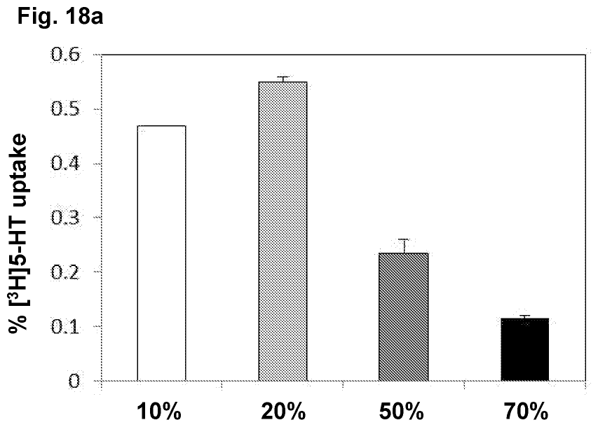

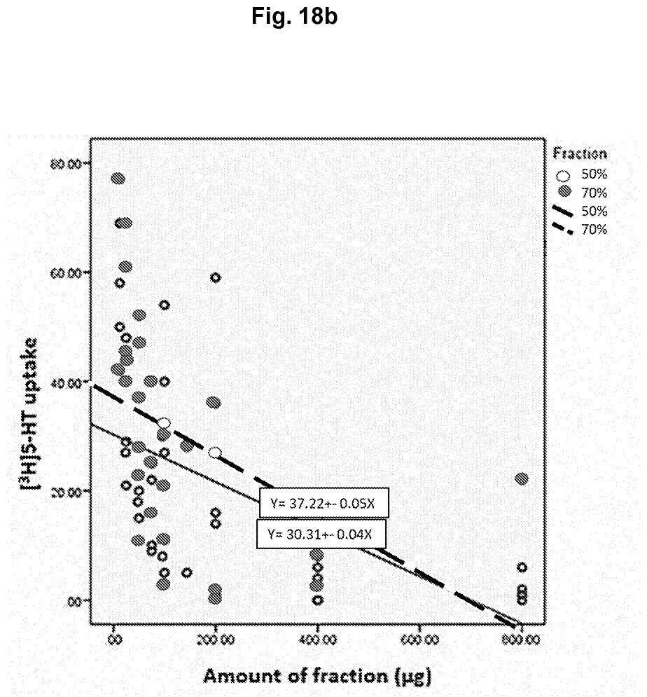

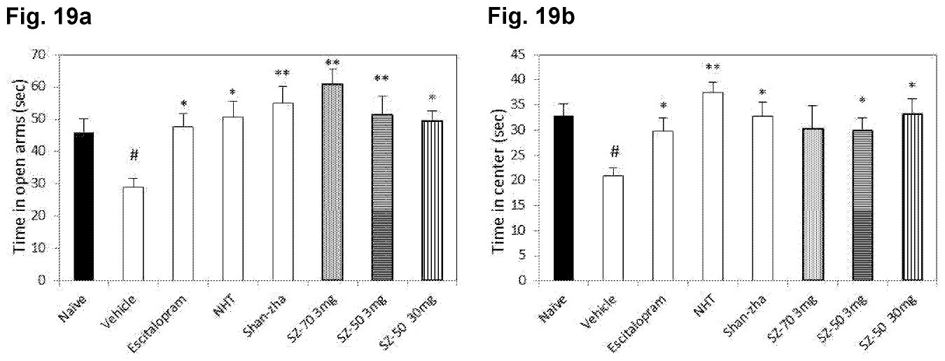

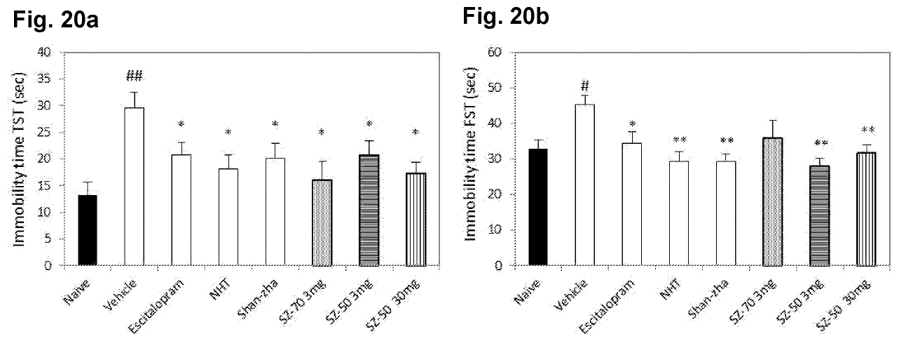

Four initial ethanol fractions were produced as described in the experimental procedures: 10%, 20%, 50% and 70% ethanol fractions. The 50% and 70% fractions (termed SZ-50 and SZ-70, respectively) were chosen for further behavioral evaluation based upon their ability to inhibit synaptosomal serotonin uptake (FIG. 18A-18B), and were found to induce anxiolytic (FIG. 19A-19B) and antidepressant effects (FIG. 20A-20B).

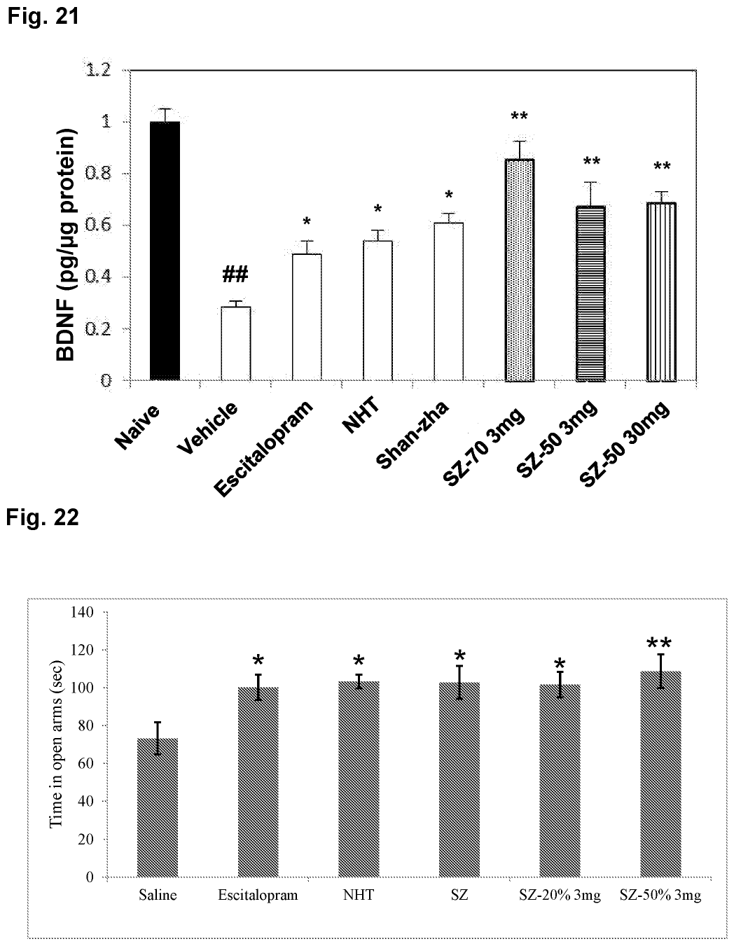

Low serotonin transporter (SERT) levels are known to be associated with depression and anxiety. Nevertheless, clinical and preclinical evidence on alterations in SERT levels following treatment with SSRIs is contradictory (Reimold, et al., Mol Psychiatry, 2008. 13(6): p. 606-13, 557). Another factor that may be involved in the mechanisms underlying the fractions' therapeutic effect is BDNF. Treatment with SZ-50 and SZ-70 fractions led to prominent increases in hippocampal BDNF levels in a similar manner to Shan-zha, NHT and escitalopram (FIG. 21).

As indicated above, SZ-50 and SZ-70 fractions exhibited the highest affinity to SERT in the serotonin uptake inhibition assay (FIG. 18A-18B). Behavioral assessment following a 3-week treatment with SZ-50 (3 and 30 mg/kg), SZ-70 (3 mg/kg) yielded the following results. All fractions and doses significantly reduced anxiety-like behavior in the EPM [F(7,139)=3.94, p<0.001] (p<0.003, p<0.001 and p<0.001, respectively) (FIG. 19A). Treatment with SZ-50 (30 mg/kg) and SZ-50 (3 mg/kg) fractions reduced anxiety-like behavior in the OFT [F(7,137)=3.452, p<0.002] (p<0.003 and p<0.037, respectively) (FIG. 19B). In addition, all fractions significantly reduced depressive-like behavior in the TST [F(7,139)=3.28, p<0.003] (p<0.002, p<0.042 and p<0.008, respectively) (FIG. 20A). Significant reductions were also found in the FST following treatment with SZ-50 (30 mg/kg) and SZ-50 (3 mg/kg) [F(7,138)=4.403, p<0.001] (p<0.001 and p<0.001, respectively) (FIG. 20B). No differences in locomotor activity in the OFT were observed [F(7,138)=1.324, p<0.243] (Data not shown). Concomitant elevations in hippocampal BDNF levels were observed following treatment with all fractions [F(7,22)=9.84, p<0.001] (p<0.001 for all fractions) (FIG. 21).

In order to evaluate anxiety-like behavior, the Elevated Plus Maze (EPM) was performed. One-way ANOVA indicated that time (seconds) spent in open arms significantly differs between the groups (F(5,101)=2.41, p=0.042) with a strong effect size (η²=0.107). A Dunnet post-hoc, with saline treatment defined as the control group, revealed that mice treated with saline spent significantly less time in open arms in comparison to NHT, escitalopram, SZ, SZ 50%-3 mg and SZ 20%-3 mg (FIG. 22).

In order to evaluate the effect of the various treatments on sexual behaviour, Cox proportional hazard regression model was used, applying sexual activity (yes/no) as status variable and time to first sexual activity as time variable. It was assumed that escitalopram treatment will have a negative influence on the sexual behaviour; hence, the treatment variable was dummy-coded so the escitalopram group was coded as a reference group which all the other treatments compared to.

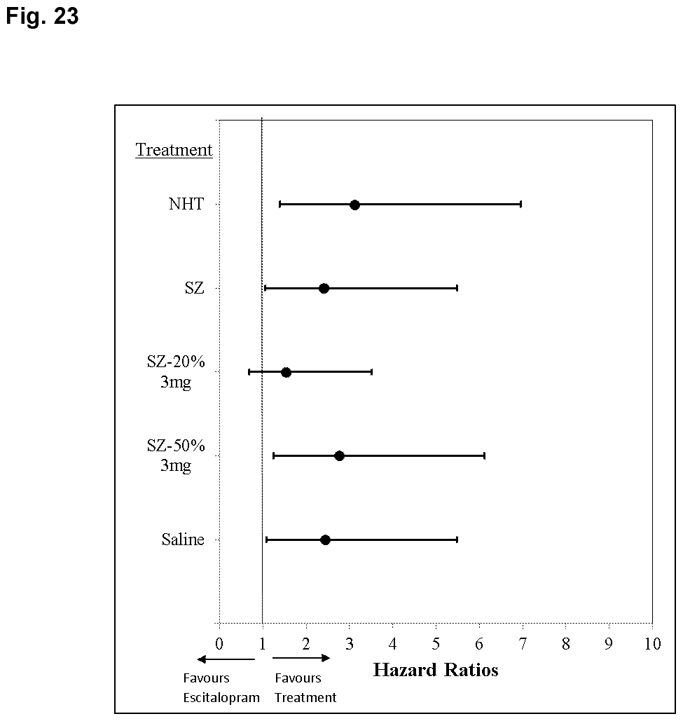

Cox regression model was found as significantly explaining mice sexual behaviour (λ² (5)=11.303, p=0.046). Specifically, the treatment variables were found as significant improvement to the model (λ² (5)=11.89, p=0.036). Hazard ratios of 95% CI and P.V for each dummy variable (i.e. comparison between escitalopram and every treatment group) were obtained. FIG. 23 shows the matching forest plot for hazard ratios.

Results indicate that NHT, SZ, SZ-50% 3 mg and saline treatment groups have significantly higher chances to show sexual behaviour in comparison to escitalopram treatment (3.11, 2.4, 2.76 and 2.43 higher chances, respectively), while SZ-20% 3 mg was not found to have significantly higher chances to demonstrate sexual behaviour comparing escitalopram (1.53 higher chances, p=0.307).

### Example 10

Genetic Analysis Following Treatment with the Shan-Zha's Fractions.

Evaluating alterations in genes and miRNAs expression levels induced by treatment with the active fraction. Brains of mice treated with the effective fraction, Shan-zha, escitalopram and vehicle collected as described above are used for the evaluation. Unbiased genome-wide transcriptomic and miRNA profiling are performed using microarrays. Genes and miRNAs that show the highest and the most significant differences are considered for further assessment. Among these genes and miRNAs a special attention is given to ITGB3, CHL1, SERT, mir-221 and mir-222. Expression levels of the involved genes and miRNAs is validated by real-time PCR.

Evaluating the behavioral and biochemical effect of treatment with the active fraction following downregulation of the gene of interest. Male mice (PND30) are subjected to UCMS for 4 weeks after which they undergo stereotaxic bilateral injection of miRNA-expressing lentiviral vector or vehicle to the hippocampus or PFC. Region and miRNA are selected based on findings as described above. Four weeks following surgery, mice are treated with the effective fraction, Shan-zha, escitalopram or vehicle for 3 weeks (n=20 mice/treatment) while on the first 3 days of treatment they are administered with 5′-bromo 2′deoxyuridine (BrdU, 100 mg/kg). Following treatment, mice undergo assessment of anxiety- and depressive-like behaviors and are sacrificed. Expression levels of genes and miRNA involved in the pathway of interest are evaluated using real-time PCR. The extent of neurogenesis and synaptogenesis are assessed using immunofluorescence.

Evaluating alterations in gene and miRNA expression levels in blood and brain in response to treatment with the active fraction. Stressed mice are treated with the effective fraction, Shan-zha, escitalopram and vehicle (n=20 mice/treatment/time point). Blood is collected from these mice as well as from vehicle-treated naïve mice prior, during (after 15 days) and after completing the treatment. Then, these mice are subjected to behavioral evaluation as described above. Genome-wide expression and miRNA profiling of the basal levels in the blood are performed. Genes and miRNAs that show the highest and the most significant differences are validated by real-time PCR. Levels of gene and miRNA of interest are correlated with the levels in blood during and after treatment completion as well as with behavioral performance in order to identify responders and non-responders. To examine whether levels of the gene and miRNA of interest in the blood reflect their levels in the brain, levels in the blood prior, throughout and after completing the treatment are correlated with levels in the brain. The tissues for this evaluation are obtained from another set of treated mice sacrificed at each time point (n=5/treatment/time point).

### Example 11

### Analysis of Monoamine Levels in the Brain

120 adolescence ICR mice (8 weeks old) were exposed to 4 weeks of unpredictable chronic mild stress (UCMS). Half of the mice (n=60) received treatment during the exposure to UCMS (starting 1 week after UCMS exposure) and half of the mice (n=60) received the treatment after the exposure to UCMS.

Mice were treated for 3 weeks with either novel herbal treatment (NHT), Shan-zha, escitalopram (CIT) or saline.

The levels of various monoamines and monoamine metabolites in the prefrontal cortex (PFC) and hippocampus were determined using HPLC.

The following monoamines were analyzed:
Serotonin system: 5HIAA (the main serotonin metabolite) as well as the 5HIAA/5HT (serotonin) ratio.
Dopamine system: 3,4-Dihydroxyphenylacetic acid (DOPAC), homovanillic acid (HVA) (both are metabolites of the neurotransmitter dopamine), dopamine (DA), as well as the DOPAC/DA ratio, the HVA/DOPAC ratio and the (DOPAC+HVA)/DA ratio.
Norepinephrine system: 3-Methoxy-4-hydroxyphenylglycol (MHPG) (a metabolite of norepinephrine degradation), norepinephrine (NE), as well as MHPG/NE ratio.
   Shan-zha was found to affect the levels of monoamines in the prefrontal cortex (PFC) and hippocampus.

1.-28. (canceled)

29. A method of treating or alleviating symptoms of anxiety disorders, stress, or depression comprising administering to a patient in need thereof an effective amount of a, pharmaceutical or nutritional composition, comprising hawthorn (shan zha, Crataegus pinnatifida) fruit, or an active fraction extracted thereof, as the active agent, wherein said composition does not comprise any additional herbal components other than hawthorn fruit or an active fraction extracted thereof, and wherein said fraction is a 20%, 50% or 70% ethanol fraction.

30. The method according to claim 29, wherein said ethanol fraction is prepared using high-performance liquid chromatography (HPLC).

31. The method according to claim 29, wherein said ethanol fraction is prepared by a method comprising:
a. homogenizing Shan-zha plant powder in 20% ethanol;
b. stirring the Shan-zha —ethanol mixture thereby obtaining an ethanol extract;
c. centrifuging the ethanol extract;
d. applying the supernatant to a separation column;
e. eluting the column with increasing concentrations of 20%, 50% or 70% ethanol; and
f. freeze-drying the eluted fractions;
thereby obtaining ethanol fractions of Shan-zha.

32. The method of claim 31, wherein said step b is performed at a temperature range of 30-80° C.

33. The method of claim 31, wherein said step d is performed by filling the entire separation column.

34. The method of claim 1 wherein said composition further comprising one or more of oil solvent, DMSO, an antioxidant, a vitamin, an inert carrier, a stabilizer, or a surfactant.

35. The method of claim 29, wherein said composition is formulated to be suitable for oral, local, or parenteral administration.

36. The method of claim 29, wherein said composition is in the form selected from the group consisting of a tablet, a capsule, a liquid, syrup, tincture, powder, granules (e.g., freeze-dried granules) and raw herbs decoction.

37. The method of claim 29, wherein said composition is encapsulated within a microcapsule.

38. The method of claim 37, wherein said microcapsule is a liposome or a micelle.

39. The method of claim 29, wherein said composition does not cause weight gain by said treated subject and/or does not result in reduction of sexual function of said treated subject.

40. The method of claim 29, wherein the amount of the composition administered is between about 1 g/day to about 15 g/day, between about 2 g/day to about 3 g/day, or about 2.5 g/day, or about 10 g/day.

41. The method of claim 29, wherein the amount of the composition administered is between about 1 mg/kg to 100 mg/kg, between about 2 mg/kg to 50 mg/kg or between about 3 mg/kg to 30 mg/kg.

42. The method of claim 29, wherein the administration of said composition causes an increase in the level of BDNF in the hippocampus and prefrontal cortex (PFC) of the treated patient, and/or does not reduce serotonin transporter (SERT) levels in the PFC of the treated patient.

43. The method of claim 29, wherein the administration of said composition does not affect the weight or the sexual function of the treated patient.

44. The method of claim 29, wherein said composition is suitable for treating breast feeding women.

45. The method of claim 29, wherein said treated patient is a breast-feeding woman.

46. The method of claim 29, wherein the efficiency of the treatment is measured by a test selected from the group consisting of the Hamilton depression rating scale (HAM), Clinical Global Impression (CGI), Sheehan Disability Scale (SDS), and a combination thereof.

47. A method for preparing an ethanol fraction of hawthorn fruit (shan za, Crataegus pinnatifida), comprising:
a. homogenizing Shan-zha plant powder in 20% ethanol;
b. stirring the Shan-zha —ethanol mixture thereby obtaining an ethanol extract;
c. centrifuging the ethanol extract;
d. applying the supernatant to a separation column;
e. eluting the column with increasing concentrations of 20%, 50% or 70% ethanol; and
f. freeze-drying the eluted fractions;
thereby obtaining ethanol fractions of Shan-zha.

48. The method of claim 48, wherein step b is performed at a temperature range of 30-80° C.
